(19) United States
(12) Patent Application Publication
(10) Pub. No.: US 2024/0052310 A1
(43) Pub. Date: Feb. 15, 2024

(54) ENHANCEMENT OF ADOPTIVE CELL TRANSFER

(71) Applicant: cellvie Inc., Houston, TX (US)

(72) Inventors: Alexander Schueller, Düsseldorf (DE); Sergej Kiprijanov, Halberstadt (DE); Nina Dumauthioz, Winterthur (CH)

(21) Appl. No.: 18/356,949

(22) Filed: Jul. 21, 2023

Related U.S. Application Data
(63) Continuation of application No. 17/221,625, filed on Apr. 2, 2021, now abandoned.
(60) Provisional application No. 63/005,167, filed on Apr. 3, 2020.

(51) Int. Cl. C12N 5/0783; A61P 35/02; A61P 35/00; A61K 35/17; A61K 35/28

(52) U.S. Cl. CPC C12N 5/0636; A61P 35/02; A61P 35/00; A61K 35/17; A61K 35/28; C12N 5/0637

(57) ABSTRACT

The disclosure relates to mitochondria-enhanced stem and immune cells, their compositions and therapeutic use.

### CROSS-REFERENCE TO RELATED APPLICATIONS

This application is a continuation of U.S. application Ser. No. 17/221,625, filed on Apr. 2, 2021, which claims priority to U.S. Provisional Application No. 63/005,167, filed on Apr. 3, 2020, each of which is hereby incorporated herein by reference in its entirety.

### FIELD

The invention relates to the field of biomedicine and specifically methods useful for therapy of cancer, infectious and autoimmune diseases. In particular, the present invention is directed to a therapeutic treatment using a mitochondria-enhanced immune effector cells, such as but not limited to chimeric antigen receptor (CAR) T-cell, CAR-NK cell, CAR-macrophage, neutrophil, tumor-infiltrating lymphocyte (TIL), gamma-delta T cell. The present invention is directed to mitochondria-enhanced stem or immune cells for use in treating cancer, infectious and autoimmune diseases. The present invention relates to increasing efficacy of cellular technologies leading to generation of immune or stem cells, which have a higher metabolic activity and/or a higher survival, in particular a higher survival in tumor hostile microenvironment. The present invention relates to increasing efficacy of cellular technologies leading to generation of higher proportions of the cytotoxic effector cells as well as to providing higher cytolytic activity of thus generated killer cells. Mitochondria transplantation can also be used for improved expansion and enhanced activity of immunosuppressive regulatory T cells for the treatment of autoimmune diseases and transplanted organ or tissue rejection. Mitochondria transplantation can also be used for improved immunosuppressive activity of regulatory T cells for the treatment of autoimmune diseases and transplanted organ or tissue rejection.

### BACKGROUND

Cancer and autoimmunity share a common origin but exert powerful forces that work in opposite directions. Both diseases result from failures in the body's immune system. Cancer often develops because the immune system failed to do its job in recognizing and/or attacking defective and/or transformed cells, allowing the cells to divide and grow. Conversely, an autoimmunity—a faulty immune response that leads to diseases such as colitis and lupus—occurs when the immune system has mistakenly attacked healthy cells. Almost any part of the body can be targeted by the immune system, including the heart, brain, nerves, muscles, connective tissues, skin, eyes, lungs, kidneys, the digestive tract, blood cells and blood vessels. A broad range of autoimmune diseases exist given that they vary according to the part of the body that is being targeted by the immune system. Common autoimmune diseases include rheumatoid arthritis, systemic lupus erythematosus, multiple sclerosis, autoimmune vasculitis, myasthenia gravis, pernicious anemia, Hashimoto's thyroiditis, type 1 diabetes, inflammatory bowel disease (IBS), autoimmune Addison's disease, Grave's disease, Sjögren's syndrome, psoriasis, and celiac diseases. To date, the American Autoimmune Related Disease Association (AARDA) has classified more than 100 autoimmune diseases, making it the third most common type of disease in the United States. In fact, autoimmune diseases affect 5 to 10% of the global population, particularly women, who are two to ten times more likely to suffer from an autoimmune disease than men. Although most diseases can occur at any age, some diseases primarily occur in childhood and adolescence (e.g. type 1 diabetes), in the mid-adult years (e.g. myasthenia gravis, multiple sclerosis), or among older adults (e.g. rheumatoid arthritis, primary systemic vasculitis) (Wang et al., 2015, “Human autoimmune diseases: a comprehensive update”, J Intern Med 278:369-95).

Cancer is also one of the leading causes of death in the developed world, with over 1.7 million cases of cancer is expected to be diagnosed in the United States alone in 2019. From this number, the projected deaths resulting from various cancers surpasses 600,000 (Seigel et al, CA Cancer J Clin. 2019; 69(1):7-34). According to the World Health Organization (WHO), cancer is a leading cause of death worldwide, accounting for an estimated 9.6 million deaths in 2018. The most common cancers are lung (2.09 million cases), breast (2.09 million cases), colorectal (1.80 million cases), prostate (1.28 million cases), skin cancer (non-melanoma; 1.04 million cases), stomach (1.03 million cases). The most common causes of cancer death are cancers of lung (1.76 million deaths), colorectal (862,000 deaths), stomach (783,000 deaths), liver (782,000 deaths), breast (627,000 deaths).

Two broad categories of T cells work together to ensure specific and long-term immunity against pathogens and tumors, whilst protecting the body from aberrant responses against self. The first subset is comprised of effector T cells and/or activated antigen-specific T cells, which eliminate pathogens and tumors. Regulatory T (Treg) cells make up the second subset and function to prevent an immune response against self. Although effector T cell responses are generally potent, a subset of infectious diseases and tumors have developed a large variety of escape mechanisms to bypass T cell control. Similarly, the incidence of autoimmune diseases, such as type 1 diabetes, highlights that Treg cells are not always successful in preventing aberrant immune responses. Moreover, in organ transplantation, Treg cells often fail to protect life-saving tissues from immune rejection.

The immune system is kept in shape through a network of signaling pathways delivered by T-cell activating receptors (accelerators) and inhibitory receptors (brakes) to regulate the balance between immune response and immune tolerance (Pardoll, 2012, “The blockade of immune checkpoints in cancer immunotherapy”, Nat Rev Cancer 12:252-64; Li et al., 2018, “Lessons learned from the blockade of immune checkpoints in cancer immunotherapy”, J Hematol Oncol 11:31). Major histocompatibility complex (MHC) molecules play key roles in the surveillance of aberrant proteins of tumor cells. T cell receptors (TCRs) on the surface of T lymphocytes recognize antigenic peptide fragments derived from these aberrant proteins in complex with MHCs. During a normal immune response, binding of these antigens to TCR, in the context of major MHC/peptide antigen presentation, initiates intracellular changes leading to T cell activation and/or recognition by the T cell of cancer cells.

In cancer patients, T cells normally build poor or no response against syngeneic transformed cells, (i) because of their poor antigenicity, (ii) for the transformed cells are not phenotypically foreign, and (iii) due to the generalized immunosuppressive conditions often associated with cancer. Therefore, adequate immune responses against tumors have seldom been observed, at least in patients treated with chemotherapeutic agents (Medler et al., 2015, “Immune response to cancer therapy: mounting an effective antitumor response and mechanisms of resistance”, Trends Cancer 1:66-75).

To counteract these deficiencies of the immune system, multiple forms of adoptive cell transfer (ACT) have recently been developed. Most prominently, Novartis introduced one of the first cancer therapies to the market—KYMRIAH® (Tisagenlecleucel), an individualized therapy that reprograms a patient's own T cells with a chimeric antigen receptor (CAR) containing a 4-1BB costimulatory domain. ACT, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park et al., 2011, “Treating cancer with genetically engineered T cells”, Trends Biotechnol 29:550-7). ACT has yet to realize its potential for treating a wide variety of diseases including cancer, infectious disease, autoimmune disease, inflammatory disease, and immunodeficiency.

Chimeric antigen receptors (CARs) are artificial receptors designed to convey antigen specificity to immune cells, such as T cells, in some instances without the requirement for MHC antigen presentation, e.g., MCH I antigen presentation, such as in CARs having an antibody derived antigen-binding domain. CARs are the transmembrane chimeric molecules with dual function, such as (i) an immune recognition of tumor antigens expressed on the surface of tumor cells; and (ii) active promotion and propagation of signaling events controlling the activation of the lytic machinery. This system allows to provide “reprogrammed T-cells” of an ex-novo activation mechanism, break the tolerance acquired by the tumor cells, and bypass restrictions of the human leukocyte antigen (HLA)/MHC-mediated antigen recognition, thus overstepping one of the barriers to a more widespread application of cellular immunotherapy.

Chimeric antigen receptor expressing T (CAR-T) cells may be used in various therapies, including cancer therapies. For example, adoptive transfer of CAR-T cells is an effective therapy for the treatment of certain hematological malignancies. The use of anti-CD19 CAR-T cells have demonstrated consistently high antitumor efficacy in children and adults affected by relapsed B-cell acute lymphoblastic leukemia (B-ALL), chronic lymphocytic leukemia (B-CLL), and non-Hodgkin lymphoma (NHL), with percentage of complete remissions ranging from 70 to 94% in different clinical trials (Wang et al., 2017, “New development in CAR-T cell therapy”, J Hematol Oncol 10:53). However, in these patients, antitumor activity is accompanied by robust CAR-T cell post-infusion expansion that is often associated with toxicity (i.e., severe cytokine-release syndrome and neurotoxicity), while patients with poor CAR-T proliferation and persistence show reduced rates of durable remissions. Thus, there is a need for CAR-T cell therapies that demonstrate CAR-T cell expansion and durable persistence following infusion while balancing CAR-T cell potency with safety.

While CAR-T cell therapies have shown efficacy in certain patient populations, CAR-T cell therapies have demonstrated limited activity in other populations, such as patients with solid tumors. CAR-T cells that enter solid tumors can stop working due to a phenomenon called T cell exhaustion. This is a state of T cell dysfunction that arises during many chronic infections and cancer. It is generally characterized by poor effector function, sustained expression of inhibitory receptors and a transcriptional state distinct from that of functional effector or memory T cells. Exhaustion can prevent optimal control of infection and tumors (Wherry, 2011, “T cell exhaustion”, Nat Immunol 12:492-9). During the development of exhaustion, CD8⁺ T cells can lose effector functions in a hierarchical manner: production of IL-2, high proliferative capacity and ex-vivo cytolytic activity are lost first, followed by functional impairments in the production of TNFα, IFNγ, beta chemokines, and degranulation, and at the most terminal stages of exhaustion these cells can be physically deleted, presumably dying due to overstimulation (Pauken and Wherry, 2015, “Overcoming T cell exhaustion in infection and cancer”, Trends Immunol 36:265-76).

There are functional challenges of engineered T cell therapy in regard to T exhaustion. Firstly, the exposure of T cells from patients to the tumor microenvironment can lead to acquiring an exhausted phenotype that causes a progression towards terminal differentiation. Upregulation of inhibitory receptors (for example, programmed cell death protein-1, PD-1) within the tumor microenvironment was shown to significantly inhibit T cell function indicating that CAR-T cells, which are produced from T cells with impaired function, might show lower effectiveness in tumor cells. Additionally, the endogenous TCR of T cells can have a negative influence on the persistence of CAR-T cells. Presence of TCR antigen when CAR is introduced into T cells with distinct TCR specificity was shown to provoke a loss in CD8⁺ CAR-T cell efficacy due to T cell exhaustion and apoptosis. Lastly, some signaling from CAR can increase differentiation and exhaustion of T cells, in that tonic CAR CD3ζ phosphorylation, triggered by antigen-independent clustering of CARs, will force early exhaustion of CAR-T cells (Kasakovski et al., 2018, “T cell senescence and CAR-T cell exhaustion in hematological malignancies”, J Hematol Oncol 11:91). Thus, there is a need for CAR-T cell therapies that reduce or eliminate T cell immune exhaustion.

Another cause of the immune system impairments is a decrease in immunological diversity of naïve T cells and an increasing number of senescent effector cells with age. This leads to a higher susceptibility to disease and potentially promotes progression of malignant tumor in elderly (van Deursen, 2014, “The role of senescent cells in ageing”, Nature 509:439-46). Many countries face demographic changes in their population with an over-proportional increase in the elderly in comparison to the young. T cell senescence impairs life-long immune protection and effective vaccination by limiting variability. T cell composition is shifted from undifferentiated naïve T cells to determined memory T cells and further to senescent T cells (Kasakovski et al., 2018, “T cell senescence and CAR-T cell exhaustion in hematological malignancies”, J Hematol Oncol 11:91). T cells in senescence tend to lose co-stimulatory molecules such as CD27 and CD28 while expressing killer cell lectin-like receptor subfamily G (KLRG-1) and CD57. Furthermore, senescent cells accumulate dysfunctional mitochondria; oxidative phosphorylation efficiency is decreased and reactive oxygen species production is increased (Korolchuk et al., 2017, “Mitochondria in Cell Senescence: Is Mitophagy the Weakest Link?”, EBioMedicine 21:7-13). An accumulation of senescent CD8⁺CD28⁻ T cells was also observed in several solid tumors, indicating the use of the suppressive activity of senescent T cells as a strategy for immune evasion. Moreover, higher numbers of CD28-CD57⁺PD-1⁺ T cells were associated with early relapse in patients with multiple myeloma (MM) after autologous stem cell transplantation (ASCT) (Chung et al., 2016, “T-cell Exhaustion in Multiple Myeloma Relapse after Autotransplant: Optimal Timing of Immunotherapy”, Cancer Immunol Res 4:61-71). In addition, senescent and exhausted T cells in patients negatively affect T cell immunotherapy. Thus, there is a need for CAR-T cell therapies that address T cell senescence.

Leukapheresis is the process by which mononuclear cells (MNCs), including subsets of T cells, are collected from a target patient, and it involves sustained blood flow through closed-loop, continuous or intermittent collection systems, and centrifugation. This is difficult in patients in advanced stages of malignancy who have low peripheral blood access. In addition, chemotherapy and radiation reduce lymphocyte count, and fewer T cells can be collected from relapsed or refractory patients who have already passed through multiple cycles of cytotoxic therapies.

Thus, the technical problem underlying the present invention is to provide new therapeutics and therapeutic strategies for inhibition of tumor-related, more specifically cancer-related T cell exhaustion as well as restoration of exhausted and senescent CAR-T cell function, such as cell survival and target-specific cytotoxicity. Furthermore, activated T lymphocytes have many times higher demand for energy than quiescent ones. The energy is primarily used for proliferation, differentiation, metabolic activity and various effector mechanisms (for example cytokine production). Therefore, rapid increase in metabolism is needed during activation of T lymphocyte. The solution of said technical problem is achieved by providing the embodiments characterized in the claims.

### SUMMARY

The present disclosure provides a composition comprising immune cells or a composition of immune cells, which are augmented by enhancer(s) comprising mitochondria, such as viable respiration-competent mitochondria, capable of enhancing or expanding an immune cell. It further provides a composition comprising immune cells or a composition of immune cells, which are augmented by enhancer(s) comprising viable respiration-competent mitochondria capable of enhancing or expanding immune cells or a population of immune cells. The immune cells may include, but are not limited to, immune cells expressing a chimeric antigen receptor (“CAR”) or artificial T-cell receptor (“TCR”) subunit, such as but not limited to CAR-T cell, CAR-NK cell, CAR-macrophage, neutrophil, tumor-infiltrating lymphocyte (TIL), gamma-delta T cell, as well as immunosuppressive Treg cells. The specification further provides compositions, including pharmaceutical compositions, comprising CAR-T cells or other engineered or propagated in vitro natural immune cells, such as tumor-infiltrating lymphocytes (TIL), natural killer (NK) cells, monocytes, macrophages or neutrophils, which are enhanced by exogenous mitochondria, such as exogenous viable respiration-competent mitochondria. The exogenous mitochondria can have different sources—e.g. autologous, autogenic, or xenogeneic. They may be freshly isolated, or previously isolated and subsequently stored until use. The sources of mitochondria may be of different nature—e.g. tissue, blood, more specifically cells circulating in the blood, or cultured cells. The described methods are based, at least in part, on the discovery that isolated mitochondria themselves, and isolated mitochondria linked to a therapeutic agent, diagnostic agent and/or imaging agent, can be delivered into target cells both in vitro and in vivo.

Provided for herein is a pharmaceutical composition comprising isolated viable mitochondria formulated in a pharmaceutically acceptable carrier in an amount effective to enhance human immune cell survival, activity, or a combination thereof relative to a human immune cell not comprising isolated viable mitochondria.

In some aspects, the mitochondria comprise eukaryotic cell mitochondria. In some aspects, the mitochondria are derived from a human cell line. In some aspects, the mitochondria are derived from a healthy donor. In some aspects, the mitochondria are derived from a patient. In some aspects, the patient is a cancer patient. In some other aspects, the patient is a patient suffering from an autoimmune disease. In some other aspects, the patient is a transplanted patient. In some other aspects, the patient is patient suffering from infectious and/or inflammatory diseases.

In some aspects, the viable mitochondria, before being formulated in a pharmaceutically acceptable carrier, have been previously isolated by using one of the isolation methods described hereinafter, each method comprising the step(s) of: (i) Isolating the mitochondria from cultured cells, tissues or organs by using Subtilisin A; or (ii) Filtrating the mitochondria through one or more filters; or (i) Isolating the mitochondria from cultured cells, tissues or organs by using Subtilisin A and subsequently (ii) filtrating the mitochondria through one or more filters.

In some aspects, the pharmaceutically acceptable carrier is formulated for delivery into a human immune cell. In some aspects, the pharmaceutically acceptable carrier is formulated for delivery into human tissues and/or organs. In some aspects, the human immune cell is autologous. In some aspects, the human immune cell is allogeneic. In some aspects, the human immune cell is a T cell, such as a CD8 T cell or a CD4 T cell, in particular a CD8 cell. In some aspects the immune cells comprises a chimeric antigen receptor (“CAR”) and/or an artificial T-Cell receptor (“TCR”) subunit or a combination thereof.

In some aspects, the composition of any one of the preceding embodiments comprises isolated viable mitochondria formulated in a pharmaceutically acceptable carrier in an amount effective to enhance the human immune cell basal oxygen consumption rate (OCR) and/or to enhance the human immune cell maximal oxygen consumption rate (OCR) relative to a human immune cell not comprising isolated viable mitochondria.

In some aspects, the composition of any one of the preceding embodiments comprises isolated viable mitochondria formulated in a pharmaceutically acceptable carrier in an amount effective to enhance the human immune cell expansion relative to a human immune cell not comprising isolated viable mitochondria.

In some aspects, the composition of any one of the preceding embodiments comprises isolated viable mitochondria formulated in a pharmaceutically acceptable carrier in an amount effective to enhance the human immune cell metabolic activity relative to a human immune cell not comprising isolated viable mitochondria.

In some aspects, the composition of any one of the preceding embodiments comprises isolated viable mitochondria formulated in a pharmaceutically acceptable carrier in an amount effective to enhance the human immune cell survival by reducing the cell exhaustion relative to a human immune cell not comprising isolated viable mitochondria.

Also provided for herein is a human stem cell or immune cell comprising exogenous mitochondria, optionally wherein the exogenous mitochondria are present in the human stem cells or in the human immune cells in an amount effective to enhance immune cell survival, activity, or a combination thereof.

In some aspects, the stem cell is an embryonic stem cell. In some aspects, the stem cell is an induced pluripotent stem cell. In some aspects, the immune cell is a pluripotent stem cell-derived immune cell. In some aspects, the immune cell is a T lymphocyte, such as a helper T cell, a cytotoxic T cell, a regulatory T cell, a memory T cell. In some aspects the T lymphocyte is a CD8 T cell. In some aspects, the T lymphocyte is a CD4 T cell or a Treg cell. In some aspects, the immune cell is a natural killer (NK) cell. In some aspects, the immune cell is a mucosal associated invariant T cell. In some aspects, the immune cell is a gamma-delta T cell. In some aspects, the immune cell is a monocyte or macrophage. In some aspects, the immune cell is a neutrophil. In some aspects, the immune cell is a B lymphocyte.

In some aspects, the stem cell or immune cell comprises a chimeric antigen receptor (“CAR”) and/or an artificial T-Cell Receptor (“TCR”) subunit. In some aspects, the stem cell or immune cell comprises an exogenous polynucleotide selected from the group consisting of a DNA, double-stranded RNA, single-stranded mRNA, and circular RNA vector encoding the CAR or the artificial TCR subunit, optionally wherein the exogenous polynucleotide is integrated into the genome of the stem cell or immune cell.

In some aspects, the CAR comprises: a. an antigen binding domain; b. a spacer domain; c. a transmembrane domain; d. optionally, a costimulatory domain; and e. an intracellular signaling domain, optionally wherein the intracellular signaling domain is an intracellular T cell signaling domain. In some aspects, the CAR comprises: a. a first CAR comprising an antigen binding domain specific for a first antigen, a spacer domain, a transmembrane domain, and a costimulatory domain and/or an intracellular T cell signaling domain; b. a cleavable domain; and c. a second CAR comprising an antigen binding domain specific for a second antigen, a spacer domain, a transmembrane domain, and a costimulatory domain and/or an intracellular T cell signaling domain.

In some aspects, the costimulatory domain is selected from the group of CD28, 4-1BB, OX40, CD27, ICOS, GITR, CD40, CD2, SLAM, and combinations thereof. In some aspects, the intracellular T cell signaling domain is selected from CD3ζ (zeta), OX40, CD27, ICOS, and combinations thereof. In some aspects, the spacer domain is selected from CH2-CH3, CD28, CD8, or combinations thereof.

In some aspects, the CAR is a multi-specific CAR comprising antigen binding domains specific for at least 2 different antigens. In some aspects, the artificial TCR comprises one or more subunits selected from the group consisting of a TCRα (alpha), a TCRβ (beta), a TCRγ (gamma), and a TCRδ (delta) subunit. In some aspects, the CAR or artificial TCR subunit is present on the cell surface.

In some aspects, the stem cell or immune cell is an allogenic or autologous stem cell or immune cell. In some aspects, the immune cell is produced from a stem cell comprising or a mesenchymal stem cell or an induced pluripotent stem cell (iPSC).

In some aspects, the stem cell or immune cell has enhanced proliferation and cytolytic activity towards to target cells.

In some aspects, the immune cell has enhanced cytolytic activity compared to a human immune cell not comprising the exogenous mitochondria.

In some aspects, the immune cell has enhanced basal oxygen consumption rate (OCR) and/or maximal oxygen consumption rate (OCR) compared to a human immune cell not comprising the exogenous mitochondria.

In some aspects, the immune cell has enhanced metabolic activity compared to a human immune cell not comprising the exogenous mitochondria.

In some aspects, the immune has enhanced expansion activity compared to a human immune cell not comprising the exogenous mitochondria.

In some aspects, the immune cell has enhanced survival, wherein the survival is enhanced by reducing the human immune cell exhaustion compared to a human immune cell not comprising the exogenous mitochondria.

In some aspects, the human stem cell or immune cell of any of the proceeding embodiments is for treating cancer, infectious, inflammatory or autoimmune disease.

Also provided for herein is a population of stem cells or immune cells, comprising any of the stem cells or immune cells described herein, respectively. In some aspects, the population of immune cells comprises NK cells, natural killer T-cells (NKT cells), macrophages, alpha/beta T cells, gamma/delta T cells, Treg cells, neutrophils or combinations thereof. In some aspects, the population of immune cells comprises CD3⁺ T cells, CD4⁺ T cells, or CD8⁺ T cells, or combinations thereof.

In some aspects, the CAR or artificial TCR subunit is introduced into the stem cells or immune cells using a virus, such as a lentivirus or adenovirus or retrovirus, nanoparticle, or a nanoparticle operably connected to a targeting moiety. In some aspects, the exogenous polynucleotide encoding the CAR and/or artificial TCR subunit is introduced into the stem cells or immune cells in vitro.

In some aspects, the population is formulated in a pharmaceutical composition in an amount effective to treat cancer in a human subject in need thereof. In some aspects, the population is formulated in a pharmaceutical composition in an amount effective to treat autoimmune diseases in a human subject in need thereof. In some aspects, the population is formulated in a pharmaceutical composition in an amount effective to treat infectious diseases in a human subject in need thereof. In some aspects, the population is formulated in a pharmaceutical composition in an amount effective to treat inflammatory diseases in a human subject in need thereof. In some aspects, the population is formulated in a pharmaceutical composition in an amount effective to treat graft vs host diseases (GvHD) in a human subject in need thereof.

In some aspects, the population of immune cells kills tumor cells more effectively and/or for longer than an equivalent population of immune cells lacking exogenous mitochondria. In some aspects, the aberrant immune responses of immune cells of the population, such as in the case of autoimmune diseases, are milder (lesser) and/or completely absent when compared to the responses of an equivalent population of immune cells lacking exogenous mitochondria.

In some aspects, the population of stem cells or immune cells comprises a CAR or artificial TCR subunit comprising an antigen binding domain specific for an antigen selected from the group: B-cell maturation antigen (BCMA, also known as tumor necrosis factor receptor superfamily member 17, TNFRSF17), CD19, CD123, CD22, CD30, CD171, CS-1 (also referred to as CD2 subset 1, CRACC, SLAMF7, CD319, and 19A24), C-type lectin-like molecule-1 (CLL-1 or CLECLI), CD33, epidermal growth factor receptor variant III (EGFRvIII), ganglioside G2 (GD2), ganglioside GD3, Tn antigen (Tn Ag or GalNAca-Ser/Thr), prostate-specific membrane antigen (PSMA), receptor tyrosine kinase-like orphan receptor 1 (ROR1), Fms-Like Tyrosine Kinase 3 (FLT3), tumor-associated glycoprotein 72 (TAG72), CD38, CD44v6, carcinoembryonic antigen (CEA), epithelial cell adhesion molecule (EpCAM), B7H3 (CD276), KIT (CD117), interleukin-13 receptor subunit alpha-2 (IL-13Ra2 or CD213A2); mesothelin, interleukin 11 receptor alpha (IL-1 IRa), prostate stem cell antigen (PSCA), protease Serine 21 (Testisin or PRSS21), vascular endothelial growth factor receptor 2 (VEGFR2), Lewis Y antigen, CD24, platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDG alp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some aspects, the population of stem cells or immune cells comprises a CAR of first, second, third or fourth generation.

Also provided for herein is a method of enhancing a population of stem cells or immune cells, optionally wherein the stem cells or immune cells express a CAR or artificial TCR subunits, comprising the steps of: a. activating stem cells or immune cells in vitro in a cell-free medium with specific activating receptor agonist antibodies capable of driving lymphocyte activation; b. exposing the stem cells or immune cells to a composition comprising isolated viable mitochondria, thereby expanding the population of stem cells or immune cells; and c. transducing the stem cells or immune cells with an exogenous polynucleotide encoding a CAR or artificial TCR subunit. In some aspects, the method comprises alternatively the step a. activating stem cells or immune cells in vitro in a cell-free medium with coated CD3/CD28 beads, optionally in presence of recombinant interleukins, such as IL-2. In some aspects, the method comprises alternatively the step b. exposing the stem cells or immune cells to a composition comprising isolated mitochondria, thereby enhancing the metabolic activity or survival or a combination thereof.

In some aspects, the stem cells or immune cells are derived from a biological sample selected from the group: blood and other liquid samples of biological origin, solid tissue samples, tissue culture of cells derived therefrom and the progeny thereof, isolated cells from biological samples. In some aspects, the specific lymphocyte activating receptor agonist is conjugated to cell mimicking cell-free supports. In some aspects, the cell-mimicking supports are paramagnetic beads.

In some aspects, the vector is a viral vector. In some aspects, the viral vector is derived from a retrovirus, lentivirus, adenovirus, adeno-associated virus, or hybrid vector.

Also provided for herein is a pharmaceutical composition for treating cancer, infectious, inflammatory or autoimmune disease comprising the stem cells or immune cells, or the population of stem cells or immune cells of any one of the preceding embodiments. In particular it is provided for herein a pharmaceutical composition for treating cancer comprising the stem cells or immune cells, or the population of stem cells or immune cells of any one of the preceding embodiments. In particular it is provided for herein a pharmaceutical composition for treating autoimmune disease comprising the stem cells or immune cells, or the population of stem cells or immune cells of any one of the preceding embodiments

The pharmaceutical composition of any one of the preceding embodiments, wherein the composition is prepared in a soluble state. In some aspects, the stem cells or immune cells are produced from viable eukaryotic cells. In some aspects, the stem cells or immune are produced in-vitro or ex-vivo. In some aspects, the stem cells or immune cells are produced through a method of autologous cell transplantation or allogeneic cell transplantation.

In some aspects, the method of allogeneic cell transplantation comprises: a. obtaining a sample of viable blood from a donor; b. separating stem cells or immune cells from the blood sample obtained in step (a); c. transducing the immune cells with one or more exogenous polynucleotides encoding CARs or artificial TCR subunits; d. optionally, contacting the stem cells or immune cells with a small molecule of any one of the preceding embodiments; and e. administering the modified stem cells or immune cells into a subject in need thereof.

In some aspects, the method of autologous cell transplantation comprises: a. obtaining a sample of viable blood taken from the subject in need; b. separating stem cells or immune cells from the blood sample obtained in step (a); c. transducing the immune cells with one or more exogenous polynucleotides encoding CARs or artificial TCR subunits; d. optionally, contacting the stem cells or immune cells with a small molecule of any one of the preceding embodiments; and e. reintroducing, e.g., reinfusing, the modified immune cells into the subject.

In some aspects, the pharmaceutical composition is administered in an amount effective to treat cancer in a human subject in need thereof. In some aspects, the pharmaceutical composition is administered in an amount effective to treat infectious disease in a human subject in need thereof. In some aspects, the pharmaceutical composition is administered in an amount effective to treat inflammatory disease in a human subject in need thereof. In some aspects, the pharmaceutical composition is administered in an amount effective to treat autoimmune disease in a human subject in need thereof.

Also provided for herein is a method of treating a subject in need thereof comprising administering to a subject the stem cells or immune cells, or population of stem cells or immune cells of any one of the preceding embodiments. In some aspects, the activity of the stem cell or immune cell is boosted by in vivo co-administration of the mitochondria comprising pharmaceutical composition of any of the preceding compositions, e.g., a pharmaceutical composition comprising isolated viable mitochondria, in particular isolated viable respiration-competent mitochondria, formulated in a pharmaceutically acceptable carrier in an amount effective to enhance human immune cell survival and/or activity relative to a human immune cell not comprising isolated viable mitochondria, or a composition comprising immune cells, optionally wherein the co-administration of the pharmaceutical composition is prior to, simultaneously, and/or following, administration of the stem cell or immune cell. In some aspects, the pharmaceutical composition is co-administered with the immune cells by intravenous infusion into the patient. In some aspects, the pharmaceutical composition is co-administered with the immune cells via intratumoral injection. In some aspects, the pharmaceutical composition is co-administered with the immune cells via intraorgan injection, or through organ-specific vasculature.

Also provided for herein is a method of treating a subject in need thereof comprising co-administering a stem cell or immune cell, or population of stem cells or immune cells, and isolated viable mitochondria, optionally wherein isolated viable mitochondria are comprised in the pharmaceutical composition of any of the preceding compositions, e.g., a pharmaceutical composition comprising isolated viable mitochondria, in particular isolated viable respiration-competent mitochondria, formulated in a pharmaceutically acceptable carrier in an amount effective to enhance human immune cell survival and/or activity relative to a human immune cell not comprising isolated viable mitochondria, or a composition comprising immune cells comprising exogenous mitochondria. In some aspects, the co-administration of the isolated viable mitochondria is prior to, simultaneously, and/or following, administration of the stem cell or immune cell.

In some aspects, the subject has a cancer selected from the group: acute lymphoblastic leukemia (ALL), acute myeloid leukemia (AML), alveolar rhabdomyosarcoma, bladder cancer (e.g., bladder carcinoma), bone cancer, brain cancer (e.g., glioblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin's lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

### BRIEF DESCRIPTION OF THE DRAWINGS

FIG. 1 displays a schematic diagram of one exemplary protocol for isolating mitochondria from tissue or cultured cells.

FIGS. 2A-2D display a dose-dependent integration of mitochondria determined using flow cytometry and fluorescence microscopy. FIG. 2A MFI of Mitotracker Red CMXRos of integrated mitochondria by flow cytometry. FIG. 2B MFI of Mitotracker Green FM of integrated mitochondria by flow cytometry. FIG. 2C Percentage of transplanted CD8⁺ T cells Mitotracker Red CMXRos+ by flow cytometry. FIG. 2D Signal of Mitotracker Red CMXRos of integrated mitochondria by fluorescence microscopy. Mitochondrial activity and mass of the stained mitochondria, which has been integrated into the CD8⁺ T cells, as seen by flow cytometry and fluorescent microscopy, are significantly increased in a dose-dependent manner in treated CD8⁺ T cells. CD8⁺ T cells from five different donors, on day 18 post activation were treated with exogenous mitochondria and analyzed 4 h post mitochondria transplantation. Dosage levels of mitochondria of 1 g, 5 g, 10 g, 20 g, and 30 g, as measured using a Qubit™ Protein Assay, were administered to 1 Million T cells. Data are representative of four independent experiments (flow cytometry) and two independent experiments (fluorescent microscopy) presented as the mean±SD of five donors (flow cytometry) and one representative donor (fluorescent microscopy). *p<0.05; **p<0.01; ***p 0.001; ****p 0.0001.

FIG. 3 displays the overall expansion of CD8⁺ T cells upon mitochondria transplantation. Mitochondria transplantation into CD8⁺ T cells was shown to enhance cellular proliferation. Overall expansion of CD8⁺ T cells. Data are representative of two pooled independent experiments and are presented as the mean±SD (six donors per group per pooled time point: 24 h, 48 h, 72 h, 140 h post transplantation, expressed in fold change compared to the control group at each time point). Resting CD8⁺ T cells between day 12 and day 18 post activation were analyzed 24 h, 48 h, 72 and 140 h post mitochondria transplantation. *p<0.05; **p<0.01; ***p<0.001; ****p<0.0001.

FIGS. 4A-4D demonstrates treated CD8⁺ T cells exhibit increased basal and maximal oxygen consumption rate. Mitochondria transplantation increases basal and maximal oxygen consumption rate (OCR) in CD8⁺ T cells. FIG. 4A Basal OCR (pmol/min). FIG. 4B Maximal respiration (OCR upon FCCP treatment-basal OCR) (pmol/min). FIG. 4C Fold change basal OCR. FIG. 4D Fold change maximal respiration (OCR upon FCCP treatment-basal OCR). Data are representative of three independent experiments and are presented as the mean±SD of two donors (7-11 technical replicates per group and per donor). *p<0.05; **p<0.01; ***p<0.001; ****p<0.0001.

FIGS. 5A-5E show assessment of mitochondrial fitness in treated and untreated CD8⁺ T cells. Mitochondria transplantation into CD8⁺ T cells was shown to enhance mitochondrial fitness. FIG. 5A MFI of Mitotracker Red CMXRos. FIG. 5B MFI of Mitotracker Green FM. FIG. 5C Fold change of mitochondrial activity. FIG. 5D Fold change of mitochondrial mass. FIG. 5E Ratio of Mitotracker Red CMXRos over Mitotracker Green FM. Data are representative of three pooled independent experiments and are presented as the mean±SD (6-11 technical replicates per group). CD8⁺ T cells, from one donor, between day 11 and day 17 post activation were analyzed 24 h post mitochondria transplantation. *p<0.05; **p<0.01; ***p<0.001; ****p<0.0001.

FIGS. 6A-6G demonstrate that mitochondria transplantation reduces T cell exhaustion. FIG. 6A MFI of LAG-3 of T cells stimulated once or four times. FIG. 6B MFI of TIM-3 of T cells stimulated once or four times. FIG. 6C MFI of LAG-3. FIG. 6D MFI of TIM-3. FIG. 6E Fold change of LAG-3.

FIG. 6F Fold change of TIM-3. FIG. 6G Percentage of non-exhausted CD8⁺ T cells post stimulations. Data represent one experiment and are presented as the mean±SD from one healthy donor (4 technical replicates). *p<0.05; **p<0.01; ***p<0.001; ****p<0.0001.

Statistical analysis: All tests were performed using GraphPad Prism software (La Jolla, CA). All data are presented as the mean+SD. Normality of the data was tested with a Shapiro-Wilk test. Comparisons between two unpaired groups were performed by parametric Student's t-test or nonparametric Mann-Whitney test. Comparisons between two paired groups not following a normal distribution were performed by nonparametric Wilcoxon matched-pairs signed rank test. For multiple comparisons, a parametric one-way analysis of variance (ANOVA) or nonparametric Krustal-Wallis test was performed (correction with Dunn's multiple comparisons test). p<0.05 were considered statistically significant (*p<0.05; **p<0.01; ***p<0.001; ****p<0.0001).

### DETAILED DESCRIPTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The term “isolated” means altered or removed from the natural state or environment. For example, a nucleic acid or a peptide naturally present in a living animal or cell is not “isolated,” but the same nucleic acid or peptide partially or completely separated from the coexisting materials of its natural state is “isolated.” As used herein, the term “isolated mitochondria” means viable mitochondria that are free of extraneous eukaryotic cell material. Isolated mitochondria can exist in substantially purified form, or can exist in a non-native environment such as, for example, an exogenous host cell.

The term “viable mitochondria” is used throughout the specification to describe viable mitochondria, which are active, functioning and respiration-competent mitochondria.

As used herein, the term “transplantation” is used throughout the specification as a general term to describe the process of implanting an organ, tissue, mass of cells, individual cells, or cell organelles into a recipient. The term “cell transplantation” is used throughout the specification as a general term to describe the process of transferring at least one cell, e.g., an enhanced immune cell described herein, to a recipient. The terms include all categories of transplants known in the art, including blood transfusions. Transplants are categorized by site and genetic relationship between donor and recipient. The term includes, e.g., autotransplantation (removal and transfer of cells or tissue from one location on a patient to the same or another location on the same subject), allotransplantation (transplantation between members of the same species), and xenotransplantation (transplantations between members of different species).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “antibody” is used herein in its broadest sense and includes certain types of immunoglobulin molecules comprising one or more antigen-binding domains that specifically bind to an antigen or epitope. The term also includes non-immunoglobulin antigen-binding protein molecules, so-called antibody mimetics. An antibody specifically includes intact antibodies (e.g., intact immunoglobulins G, IgG), antibody fragments (e.g., Fab fragment, single-chain Fv (scFv), single domain antibodies, V_{H}, V_{L}, V_{HH}, NAR, tandem scFvs, diabodies, single-chain diabodies, DARTs, tandAbs, minibodies, single-domain antibodies (e.g., camelid V_{HH}), other antibody fragments or formats known to those skilled in the art), and antibody mimetics (e.g., adnectins, affibodies, affilins, anticalins, avimers, DARPins, knottins, etc.). The antibodies can be monospecific, bi- and multi-specific.

The term “antigen-binding domain” means the portion of an antibody or T cell receptor that is capable of specifically binding to an antigen or epitope via a variable domain. One example of an antigen-binding domain is an antigen-binding domain formed by an interface of the variable domains, V_{H} and V_{L}, of an antibody heavy and light chain, respectively. Another example of an antigen-binding domain is an antigen-binding domain formed by diversification of certain loops from the antibody mimetics. Another example of an antigen-binding domain is the variable domains of a TCR, such as TCR domains containing CDRs, e.g., αCDR1, αCDR2, αCDR3, βCDR1, βCDR2, and βCDR3.

As used herein, “variable domain” refers to a variable nucleotide sequence that arises from a recombination event, for example, it can include a V, J, and/or D region of a T cell receptor (TCR) sequence from a T cell, such as an activated T cell, or it can include a V, J, and/or D region of an antibody. The term “antigen-binding fragment” refers to at least one portion of an antibody or TCR, or recombinant variants thereof, that contain the antigen binding domain, i.e., variable domains and hypervariable loops, so-called complementarity determining regions (CDRs), that are sufficient to confer recognition and specific binding of the antigen-binding fragment to a target, such as an antigen and its defined epitope. Examples of antigen-binding fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either V_{L} or V_{H}), camelid V_{HH} domains (nanobodies), multi-specific antibodies generated from antibody fragments, and TCR fragments. Exemplary antibody and antibody fragment formats are described in detail in Brinkmann et al. (MABS, 2017, Vol. 9, No. 2, 182-212), herein incorporated by reference for all that it teaches.

The term “scFv” refers to a fusion protein comprising a variable fragment of the antibody heavy chain (V_{H}) linked in its C-terminus with an N-terminus of a variable fragment of the antibody light chain (V_{L}) via a flexible peptide linker, and capable of being expressed as a single polypeptide chain, and wherein the scFv retains the specificity of the intact antibody from which it is derived.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Gly-Ser)n, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₃ or (Gly₄Ser)₄. In another embodiment, the linkers include multiple repeats of (Gly₂Ser), (GlySer) or (Gly₃Ser). Also included within the scope of the invention are linkers described in WO2012/138475 (incorporated herein by a reference).

“Heavy chain variable region” or “V_{H}” (or, in the case of the camelid single domain antibodies, e.g., nanobodies, “V_{HH}”) with regard to an antibody refers to the fragment of the heavy chain that contains three CDRs interposed between flanking stretches known as framework regions (FR); these framework regions are generally more conserved than the CDRs and form a scaffold to support the CDRs.

Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The term “antibody heavy chain,” refers to the larger of the two types of polypeptide chains present in an antibody molecule in their naturally occurring conformations, and which typically determines the immunoglobulin class to which the antibody belongs.

The term “antibody light chain,” refers to the smaller of the two types of polypeptide chains present in antibody molecules in their naturally occurring conformations. Kappa (“κ”) and lambda (“λ”) light chains refer to the two major antibody light chain isotypes.

The term “recombinant antibody” refers to an antibody that is generated using recombinant DNA technology, such as, for example, an antibody expressed by a bacterial, yeast, plant or mammalian cell. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response, for example, when the antigen is processed by an antigen-presenting cell (APC). This immune response may involve either antibody production, or the activation of specific immunologically competent cells, or both.

A person skilled in the art will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated by a chemical synthesis; it can also be derived from a biological sample, or might be a macromolecule besides a polypeptide, e.g., lipid or carbohydrate. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

“Humanized” forms of non-human (e.g., murine) antibodies are immunoglobulins, which contain fully human constant domains (e.g., Cκ or Cλ, C_{H}1, C_{H}2, C_{H}3) and the variable domains (V_{H}, V_{L}) with minimal sequences derived from non-human immunoglobulins. For the most part, humanized antibodies, TCRs, and the antigen-binding fragments thereof are human immunoglobulins (e.g., recipient antibody, TCR, or antigen-binding fragment) in which residues from a CDR of the recipient are fully or partly replaced by residues from a CDR of a non-human species (donor antibody or TCR), such as mouse, rat, rabbit, chicken, or non-human primate antibody, having the desired specificity, affinity, and functional activity (e.g., blocking receptor-ligand interaction). In some instances, some residues of the V_{H} or V_{L} framework region (FR) of the human immunoglobulin are replaced by corresponding non-human residues, in order to restore the humanized antibody binding activity. Furthermore, a humanized antibody/TCR/antigen-binding fragment can comprise residues, which are found neither in the recipient antibody nor in the imported CDR or framework sequences. These modifications can further refine and optimize antibody, TCR, or antigen-binding fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” or “human TCR” is one which possesses an amino acid sequence corresponding to that of an antibody produced by a human or a human cell, or derived from a non-human source that utilizes a human antibody or TCR repertoire or human antibody/TCR-encoding sequences (e.g., obtained from human sources or designed de novo). Human antibodies and TCRs specifically exclude humanized antibodies and TCRs, respectively.

With regard to the binding of an antibody, TCR, or antigen-binding fragment thereof to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody, TCR, or antigen-binding fragment thereof to the target molecule is competitively inhibited by the control molecule. Specific binding, as used herein, can refer to an affinity in which the K_{D} value is below 10⁻⁶M, 10⁻⁷M, 10⁻⁸M, 10⁻⁹M, or 10⁻¹⁰M. Affinity can be measured by common methods known in the art, including those described herein, such as surface plasmon resonance (SPR) technology (e.g., BIACORE©) or biolayer interferometry (e.g., FORTEBIO©).

The term “autologous” refers to any material derived from the same individual to whom it is later to be re-introduced into the individual.

The term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “chimeric antigen receptor” or “CAR” refers to a recombinant polypeptide derived from the various polypeptides comprising an antigen-binding moiety (e.g., a polypeptide having at least an antigen-binding domain or antigen-binding fragment thereof) fused to a primary cytoplasmic signaling sequence (also referred to as a “primary signaling domain”) that acts in a stimulatory manner and that may contain a signaling motif which is known as immunoreceptor tyrosine-based activation motif or “ITAM”. Examples of an ITAM containing primary cytoplasmic signaling sequence that is of particular use in the invention includes, but is not limited to, those derived from CD3ζ (zeta), FcRγ (gamma), FcRβ (beta), CD3γ, CD3δ (delta), CD3ε (epsilon), CD5, CD22, CD79a, CD79b, CD278 (also known as “ICOS”) and CD66d. A CAR provides typically provides an engineered immune cell, such as a T lymphocyte, with antibody-type specificity or TCR-type specificity and activates some or all the functions of an effector cell, including the production of IL-2 and lysis of the target cells following signaling in T cells.

The antigen-binding domain or antigen-binding fragment thereof of the CARs described herein may exist in a variety of forms, for example where the antigen-binding domain is expressed as part of a contiguous polypeptide chain including, for example, a single domain antibody fragment (sdAb) or heavy chain antibody (HCAb), a single-chain Fv antibody (scFv), either naturally-derived, or synthetic, which binds to an antigen. The antigen-binding domain or antigen-binding fragment thereof of the CARs described herein can include any of the antibody formats or antibody fragment formats described herein. The antigen-binding domain or antigen-binding fragment thereof of the CARs described herein can include sequences that are not derived from antibodies, including but not limited to chimeric or artificial T-cell receptors (TCR). These chimeric/artificial TCRs may comprise a polypeptide sequence that recognizes a target antigen, where the recognition sequence may be, for example, but not limited to, the recognition sequence derived from a TCR or an scFv. The intracellular domain polypeptides are those that act to activate the T cell. Chimeric/artificial TCRs are discussed in, for example, Gross, G., and Eshhar, Z., FASEB Journal 6:3370-3378 (1992), and Zhang, Y., et al., PLOS Pathogens 6: 1-13 (2010).

A “CAR-T cell” is a T cell that has been transduced according to the methods disclosed herein and that expresses a CAR gene, e.g., incorporated randomly into the genome or purposely integrated into the CCR5 and AAVS1 loci, or into the T-cell receptor a constant (TRAC) locus. In some embodiments, the T cell is a CD4⁺ T cell, a CD8⁺ T cell, or a CD4⁺/CD8⁺ T cell. In some embodiments, the T cell is a regulatory T cell. In some embodiments, the T cell is autologous, allogeneic, or xenogeneic with reference to a subject.

As used herein, the term “subject” means a mammalian subject. Exemplary subjects include humans, monkeys, dogs, cats, mice, rats, cows, horses, camels, goats, rabbits, and sheep. In certain embodiments, the subject is a human. A “patient” is a subject suffering from or at risk of developing a disease, disorder or condition or otherwise in need of the compositions and methods provided herein.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented, at least over a period of time, in that individual.

As used herein, the term “CD19”, B-lymphocyte antigen CD19, CD19 molecule (Cluster of Differentiation 19), B-Lymphocyte Surface Antigen B4, T-Cell Surface Antigen Leu-12 and CVID3 is a transmembrane protein that in humans is encoded by the gene CD19. In humans, CD19 is expressed in all B lineage cells, except for plasma cells, and in follicular dendritic cells. CD19 plays two major roles in human B cells. It acts as an adaptor protein to recruit cytoplasmic signaling proteins to the membrane and it works within the CD19/CD21 complex to decrease the threshold for B cell receptor signaling pathways. Due to its presence on all B cells, it is a biomarker for B lymphocyte development, lymphoma diagnosis and can be utilized as a target for leukemia and lymphoma immunotherapies.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “cytotoxic agent,” as used herein, refers to a substance that inhibits or prevents a cellular function and/or causes cell death or destruction.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematological malignancy (blood tumor).

The term “pharmaceutical composition” refers to a preparation which is in such form as to permit the biological activity of an active ingredient and/or maintain or improve viability of a biological entity (e.g., a cell) contained therein to be effective in treating a subject, and which contains no additional components, which are unacceptably toxic to the subject in the amounts provided in the pharmaceutical composition.

The term “pharmaceutically acceptable carrier” includes saline, solvents, dispersion media, coatings, antibacterial and antifungal agents, isotonic and absorption delaying agents, and the like, compatible with pharmaceutical administration. In some embodiments, the pharmaceutically acceptable carrier is phosphate buffered saline, saline, Krebs buffer, Tyrode's solution, contrast media, or omnipaque, or a mixture thereof. The term “pharmaceutically acceptable carrier” includes also sterile mitochondria buffer (300 mM sucrose; 10 mM K+-HEPES (potassium buffered (4-(2-hydroxyethyl)-1-piperazineethanesulfonic acid, pH 7.2); 1 mM K+-EGTA, (potassium buffered ethylene glycol tetraacetic acid, pH 8.0)). The term further includes a respiration buffer (250 mM sucrose, 2 mM KH2PO4, 10 mM MgCh, 20 mM K-15 HEPES Buffer (pH 7.2), and 0.5 mM K-EGTA (pH 8.0)). The term further includes a T cell medium, e.g. RPMI 1640 medium GlutaMAX™ Supplement 500 ml (ThermoFisher, 61870010).

The terms “modulate” and “modulation” refer to reducing or inhibiting or, alternatively, activating or increasing, a recited variable.

The terms “increase” and “activate” refer to an increase of 5%, 10%, 15%, 20%, 25%, 30%, 35%, 40%, 45%, 50%, 55%, 60%, 65%, 70%, 75%, 80%, 85%, 87%, 90%, 95%, 98%, 99%, 100%, 1.5-fold, 2-fold, 2.5-fold, 3-fold, 3.5-fold, 4-fold, 4.5-fold, 5-fold, 5.5.-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The term “effector T cell” includes T helper (i.e., CD4⁺) cells and cytotoxic (i.e., CD8⁺) T cells. CD4⁺ effector T cells typically contribute to the development of several immunologic processes, including maturation of B cells into plasma cells and memory B cells, and activation of cytotoxic T cells and macrophages. CD8⁺ effector T cells typically destroy virus-infected cells and tumor cells. See Seder and Ahmed, 2003, incorporated by reference in its entirety, for additional information on effector T cells (Seder and Ahmed, 2003, “Similarities and differences in CD4⁺ and CD8⁺ effector and memory T cell generation”, Nat Immunol 4:835-42).

The term “regulatory T cell” or “Treg” includes cells that regulate immunological tolerance, for example, by suppressing effector T cells. In some aspects, the regulatory T cell has a CD4⁺CD25⁺Foxp3⁺ phenotype. In some aspects, the regulatory T cell has a CD8⁺CD25⁺ phenotype. See Nocentini et al., Br. J. Pharmacol., 2012, 165:2089-2099, incorporated by reference in its entirety, for additional information on regulatory T cells.

The term “dendritic cell” refers to a professional antigen-presenting cell capable of activating a naïve T cell and stimulating growth and differentiation of a B cell.

The phrase “disease associated with expression of [target]” includes, but is not limited to, a disease associated with expression of [target] or condition associated with cells which express [target] including, e.g., proliferative diseases such as a cancer or malignancy or a precancerous condition. In one aspect, the cancer is a mesothelioma. In one aspect, the cancer is a pancreatic cancer. In one aspect, the cancer is an ovarian cancer. In one aspect, the cancer is a gastric cancer. In one aspect, the cancer is a lung cancer. In one aspect, the cancer is an endometrial cancer. Non-cancer related indications associated with expression of [target] include, but are not limited to, e.g., autoimmune disease, (e.g., lupus, rheumatoid arthritis, colitis), inflammatory disorders (allergy and asthma), and transplantation.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation” refers to a primary response induced by binding of a stimulatory domain or stimulatory molecule (e.g., a CAR or a TCR/CD3 complex) with its cognate ligand or antigen-independent CD3/CD28 beads when in vitro, thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell or an engineered immune cell (e.g., an immune cell engineered to express a CAR) that provides the primary cytoplasmic signaling sequence(s) that regulate primary activation of a TCR/CAR complex in a stimulatory way for at least some aspect of a signaling pathway, such as a T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. In one aspect, the primary signal is initiated by, for instance, binding of a CAR (e.g., an antibody fragment or chimeric TCR) to its cognate antigen or epitope.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs typically process antigens and present them to T cells, but may also be “loaded” with preprocessed antigenic peptides.

An “intracellular signaling domain,” as the term is used herein, refers to an intracellular portion of a molecule involved in generating a signal that promotes an immune effector function, such as the effector function of a TCR- or CAR-expressing T cell. Examples of immune effector function, e.g., in a CAR-expressing T cell, include cytolytic activity and T helper cell activity, including the secretion of cytokines. In an embodiment, the intracellular signaling domain can comprise a primary cytoplasmic signaling sequence. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that may be required for an efficient immune response. Costimulatory molecules include, but are not limited to, an MHC class I molecule, BTLA and a Toll ligand receptor, as well as DAP10, DAP12, CD30, LIGHT, OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1nn” refers to a member of the TNFR superfamily with an amino acid sequence provided as GenBank Acc. No. AAA62478.2, or the equivalent residues from a non-human species, e.g mouse, rodent, monkey, ape and the like; and a “4-1BB costimulatory domain” is defined as amino acid residues 214-255 of GenBank Acc. No. AAA62478.2, or equivalent residues from non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain one or more introns.

The term “endogenous” refers to any material from or produced inside an organism, cell, tissue or system.

The term “exogenous” refers to any material introduced from or produced outside an organism, cell, tissue or system. In case of a patient, the term “exogenous” may refer to patient-, donor- or cell culture-derived material. For example, mitochondria isolated from the patients' muscle tissue and subsequently introduced to a population of immune cells, which may be autologous to the patient or autogenic, are considered exogenous. The term “exogenous mitochondria” refers to any mitochondria isolated from an autogenous source, an allogeneic source, and/or a xenogeneic source, wherein the source's nature may be of tissue, blood, or cultured cells.

The term “expression” refers to the transcription and/or translation of a particular nucleotide sequence driven by a promoter.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it does not need to be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct. As used herein with reference to a disease, disorder or condition, the terms “treatment”, “treat”, “treated”, or “treating” refer to prophylaxis and/or therapy.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein encoding regions, are in the same reading frame.

The term “parenteral” administration of an immunogenic composition includes, e.g., subcutaneous (s.c.), intravenous (i.v.), intramuscular (i.m.), intranasal or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR) and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

The term “promoter” refers to a DNA sequence recognized by the transcription machinery of the cell, or introduced synthetic machinery, that can initiate the specific transcription of a polynucleotide sequence.

The term “promoter/regulatory sequence” refers to a nucleic acid sequence which can be used for expression of a gene product operably linked to the promoter/regulatory sequence. In some instances, this sequence may be the core promoter sequence and in other instances, this sequence may include an enhancer sequence and other regulatory elements, which are required for expression of the gene product. The promoter/regulatory sequence may, for example, be one, which expresses the gene product in a tissue specific manner.

The term “constitutive promoter” refers to a nucleotide sequence which, when operably linked with a polynucleotide which encodes or specifies a gene product, causes the gene product to be produced in a cell under most or all physiological conditions of the cell.

The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, a 5′-cap (also termed an RNA cap, an RNA 7-methylguanosine cap or an RNA m7G cap) is a modified guanine nucleotide that has been added to the “front” or 5′-end of a eukaryotic messenger RNA shortly after the start of transcription. The 5′ cap consists of a terminal group, which is linked to the first transcribed nucleotide. Its presence is critical for recognition by the ribosome and protection from RNAses. Cap addition is coupled to transcription, and occurs co-transcriptionally, such that each influences the other. Shortly after the start of transcription, the 5′-end of the mRNA being synthesized is bound by a cap-synthesizing complex associated with RNA polymerase. This enzymatic complex catalyzes the chemical reactions that may be required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the poly(A) is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell, which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” refers to antigens that are common to specific hyperproliferative disorders. In certain aspects, the hyperproliferative disorder antigens of the present invention are derived from, cancers including but not limited to primary or metastatic melanoma, mesothelioma, renal cell carcinoma, stomach cancer, breast cancer, lung cancer, ovarian cancer, prostate cancer, colon cancer, cervical cancer, brain cancer, liver cancer, pancreatic cancer, kidney, endometrial, and stomach cancer.

In some instances, the disease is a cancer selected from the group consisting of targeted and tailored specific cancer list, which can include: mesothelioma, papillary serous ovarian adenocarcinoma, clear cell ovarian carcinoma, mixed Mullerian ovarian carcinoma, endometroid mucinous ovarian carcinoma, malignant pleural disease, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with targeted expression, and any combination thereof.

The term “transfected” or “transformed” or “transduced” refers to a process by which exogenous nucleic acid is transferred or introduced into the host cell. A “transfected” or “transformed” or “transduced” cell is one, which has been transfected, transformed or transduced with exogenous nucleic acid. The cell includes the primary subject cell and its progeny.

The term “T cell exhaustion” and “exhausted T cell” refers to either hyporesponsive T cells or “dysfunctional” T cells.

The term “to enhance cell survival, activity, or combination thereof”, or “to enhance cell survival or activity, or a combination thereof” refers to the enhancement of one of the two or both cell features. The term “activity” refers to cell effector function, such as cytotoxic activity towards the target cell expressing a certain antigen and detected by the TCR specific for that antigen or cytokine production. It further refers to metabolic activity, proliferative capacity and ability to expand and divide, capacity to resist to exhaustion, and suppressive activity.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Enhanced Immune Cells and Compositions Related Thereto

The present disclosure contemplates a composition comprising cells (e.g., stem cells and/or immune cells, such as, but not limited to, αβT cells, γδT cells, Treg cells, CAR-T cells, such as CD4 CAR-T cells or CD8 CAR-T cells, NK cells, CAR-NK cells, NK T cells, macrophages, CAR-macrophages, neutrophils, CAR-neutrophils, etc.), wherein the cells comprise exogenous mitochondria. The exogenous mitochondria may be autologous mitochondria, allogeneic mitochondria, xenogeneic mitochondria, encapsulated mitochondria, or autogenous or autologous mitochondria with genetic modification.

Accordingly, provided for herein is a pharmaceutical composition comprising isolated viable mitochondria formulated in a pharmaceutically acceptable carrier in an amount effective to enhance human immune cell survival, activity, or a combination thereof.

In some embodiments, the pharmaceutical composition comprises isolated viable mitochondria formulated in a pharmaceutically acceptable carrier in an amount effective to enhance the human immune cell basal OCR or maximal OCR relative to a human immune cells not comprising mitochondria. In some embodiments, the enhancement of the human immune cell basal OCR or maximal OCR is in the range (expressed in folds) of between 1.1-fold to 100-fold, such as 1.1 to 99, 1.1 to 90, 1.1 to 80, 1.1 to 70, 1.1 to 60, 1.1 to 50, 1.1 to 40, 1.1 to 30, 1.1 to 20, 1.1 to 10, 1.1 to 5, 1.1 to 2, 1.1 to 1.8, 1.1 to 1.5, 1.2 to 99, 1.2 to 90, 1.2 to 80, 1.2 to 70, 1.2 to 60, 1.2 to 50, 1.2 to 20, 1.2 to 10, 1.2 to 5, 1.2 to 2.5, 1.3 to 90, 1.3 to 80, 1.3 to 70, 1.3 to 50, 1.3 to 40, 1.3 to 30, 1.3 to 20, 1.3 to 10, 1.3 to 5, 1.3 to 1.5, 1.4 to 100, 1.4 to 95, 1.4 to 90, 1.4 to 80, 1.4 to 70, 1.4 to 60, 1.4 to 50, 1.4 to 30, 1.4 to 25, 1.4 to 20, 1.4 to 10, 1.4 to 5, 1.4 to 3, 1.4 to 2.5, 1.5 to 99, 1.5 to 95, 1.5 to 90, 1.5 to 80, 1.5 to 70, 1.5 to 60, 1.5 to 50, 1.5 to 50, 1.5 to 40, 1.5 to 30, 1.5 to 20, 1.5 to 10, 1.5 to 5, 1.5 to 2.5, 2 to 99, 2 to 90, 2 to 80, 2 to 70, 2 to 60, 2 to 50, 2 to 40, 2 to 35, 2 to 30, 2 to 20, 2 to 10, 2 to 5, 2 to 4, 2 to 2.5, 3 to 99, 3 to 90, 3 to 80, 3 to 70, 3 to 60, 3 to 50, 3 to 40, 3 to 30, 3 to 25, 3 to 20, 3 to 10, 4 to 99, 4 to 80, 4 to 70, 4 to 60, 4 to 50, 4 to 55, 4 to 25, 4 to 20, 4 to 15, 4 to 10, 5 to 100, 5 to 80, 5 to 50, 5 to 30, 5 to 20, 5 to 10, 5.5 to 9, 5.5 to 7, 10 to 90, 10 to 50, 10 to 20, 20 to 100, 20 to 50, 25 to 40, 20 to 35, 30 to 100, 30 to 50, 40 to 100, 40 to 70, 40 to 60, 40 to 50, 50 to 100, 50 to 90, 50 to 80, 50 to 70, 55 to 65, 60 to 80, 75 to 90, 75 to 100, 80 to 90, 80 to 85, 85 to 100.

In some embodiments, the pharmaceutical composition comprises isolated viable mitochondria formulated in a pharmaceutically acceptable carrier in an amount effective to enhance the human immune cell expansion relative to a human immune cell not comprising isolated viable mitochondria. In some embodiments, the enhancement of the human immune cell expansion is in a the range (expressed in folds) of between 1.1 to 20 fold, such as: 1.1 to 1.5, 1.1 to 2.0, 1.1 to 3.0, 1.1 to 5.0, 1.1 to 10, 1.1 to 15, 1.1 to 20, 1.3 to 1.5, 1.3 to 2.0, 1.3 to 3.0, 1.3 to 5.0, 1.3 to 10, 1.3 to 15, 1.3 to 20, 1.5 to 2.0, 1.5 to 3.0, 1.5 to 5.0, 1.5 to 10, 1.5 to 15, 1.5 to 20, 2.0 to 3.0, 2.0 to 5.0, 2.0 to 10, 2.0 to 15, 2.0 to 20, 3.0 to 5.0, 3.0 to 10, 3.0 to 15, 3.0 to 20, 4.0 to 5.0, 4.0 to 10, 4.0 to 15, 4.0 to 20, 5.0 to 10, 5.0 to 15, 5.0 to 20, 6.0 to 10, 6.0 to 15, 6.0 to 20, 7.0 to 10, 7.0 to 15, 7.0 to 20, 8.0 to 10, 8.0 to 15, 8.0 to 20, 9.0 to 10, 9.0 to 15, 9.0 to 20, 10 to 15, 10 to 20, 11 to 15, 11 to 20, 12 to 15, 12 to 20, 13 to 15, 13 to 20, 14 to 15, 14 to 20, 15 to 20, 16 to 20, 17 to 20, 18 to 20, 19 to 20.

In some embodiments, the pharmaceutical composition comprises isolated viable mitochondria formulated in a pharmaceutically acceptable carrier in amount effective to enhance the human immune cell metabolic activity relative to a human cell not comprising isolated mitochondria. In some embodiment, the enhancement of the metabolic activity is in the range (expressed in folds) of between 1.1-fold to 100-fold, such as 1.1-fold to 100-fold, such as 1.1 to 99, 1.1 to 90, 1.1 to 80, 1.1 to 70, 1.1 to 60, 1.1 to 50, 1.1 to 40, 1.1 to 30, 1.1 to 20, 1.1 to 10, 1.1 to 5, 1.1 to 2, 1.1 to 1.8, 1.1 to 1.5, 1.2 to 99, 1.2 to 90, 1.2 to 80, 1.2 to 70, 1.2 to 60, 1.2 to 50, 1.2 to 20, 1.2 to 10, 1.2 to 5, 1.2 to 2.5, 1.3 to 90, 1.3 to 80, 1.3 to 70, 1.3 to 50, 1.3 to 40, 1.3 to 30, 1.3 to 20, 1.3 to 10, 1.3 to 5, 1.3 to 1.5, 1.4 to 100, 1.4 to 95, 1.4 to 90, 1.4 to 80, 1.4 to 70, 1.4 to 60, 1.4 to 50, 1.4 to 30, 1.4 to 25, 1.4 to 20, 1.4 to 10, 1.4 to 5, 1.4 to 3, 1.4 to 2.5, 1.5 to 99, 1.5 to 95, 1.5 to 90, 1.5 to 80, 1.5 to 70, 1.5 to 60, 1.5 to 50, 1.5 to 50, 1.5 to 40, 1.5 to 30, 1.5 to 20, 1.5 to 10, 1.5 to 5, 1.5 to 2.5, 2 to 99, 2 to 90, 2 to 80, 2 to 70, 2 to 60, 2 to 50, 2 to 40, 2 to 35, 2 to 30, 2 to 20, 2 to 10, 2 to 5, 2 to 4, 2 to 2.5, 3 to 99, 3 to 90, 3 to 80, 3 to 70, 3 to 60, 3 to 50, 3 to 40, 3 to 30, 3 to 25, 3 to 20, 3 to 10, 4 to 99, 4 to 80, 4 to 70, 4 to 60, 4 to 50, 4 to 55, 4 to 25, 4 to 20, 4 to 15, 4 to 10, 5 to 100, 5 to 80, 5 to 50, 5 to 30, 5 to 20, 5 to 10, 5.5 to 9, 5.5 to 7, 10 to 90, 10 to 50, 10 to 20, 20 to 100, 20 to 50, 25 to 40, 20 to 35, 30 to 100, 30 to 50, 40 to 100, 40 to 70, 40 to 60, 40 to 50, 50 to 100, 50 to 90, 50 to 80, 50 to 70, 55 to 65, 60 to 80, 75 to 90, 75 to 100, 80 to 90, 80 to 85, 85 to 100.

In some embodiments, the pharmaceutical composition comprises isolated viable mitochondria formulated in a pharmaceutically acceptable carrier in amount effective to enhance the human immune survival by reducing the cell exhaustion relative to a human cell not comprising isolated mitochondria. In some embodiments, the reduction of the cell exhaustion is in the range of 0.5% to 100%, such as: 0.5% to 1.5%, 0.5% to 2.0%, 0.5% to 3.0%, 0.5% to 5.0%, 0.5% to 10%, 0.5% to 15%, 0.5% to 20%, 0.5% to 30%, 0.5% to 40%, 0.5% to 50%, 0.5% to 60%, 0.5% to 70%, 0.5% to 80%, 0.5% to 90%, 0.5% to 100%, 1.5% to 2.0%, 1.5% to 3.0%, 1.5% to 5.0%, 1.5% to 10%, 1.5% to 15%, 1.5% to 20%, 1.5% to 30%, 1.5% to 40%, 1.5% to 50%, 1.5% to 60%, 1.5% to 70%, 1.5% to 80%, 1.5% to 90%, 1.5% to 100%, 2.0% to 3.0%, 2.0% to 5.0%, 2.0% to 10%, 2.0% to 15%, 2.0% to 20%, 2.0% to 30%, 2.0% to 40%, 2.0% to 50%, 2.0% to 60%, 2.0% to 70%, 2.0% to 80%, 2.0% to 90%, 2.0% to 100%, 5.0% to 10%, 5.0% to 15%, 5.0% to 20%, 5.0% to 30%, 5.0% to 40%, 5.0% to 50%, 5.0% to 60%, 5.0% to 70%, 5.0% to 80%, 5.0% to 90%, 5.0% to 100%, 10% to 15%, 10% to 20%, 10% to 30%, 10% to 40%, 10% to 50%, 10% to 60%, 10% to 70%, 10% to 80%, 10% to 90%, 10% to 100%, 20% to 30%, 20% to 40%, 20% to 50%, 20% to 60%, 20% to 70%, 20% to 80%, 20% to 90%, 20% to 100%, 30% to 40%, 30% to 50%, 30% to 60%, 30% to 70%, 30% to 80%, 30% to 90%, 30% to 100%, 40% to 50%, 40% to 60%, 40% to 70%, 40% to 80%, 40% to 90%, 40% to 100%, 50% to 60%, 50% to 70%, 50% to 80%, 50% to 90%, 50% to 100%, 60% to 70%, 60% to 80%, 60% to 90%, 60% to 100%, 70% to 80%, 70% to 90%, 70% to 100%, 80% to 90%, 80% to 100%, 90% to 100%.

Accordingly, also provided for herein are populations of human immune cells comprising exogenous mitochondria, such as but not limited to autologous mitochondria, allogeneic mitochondria, genetically engineered mitochondria, and mitochondria encapsulated by a liposome or coupled to specific agents. These cells can be either any effector cells known in the art with anti-tumor activity or immunosuppressive immune cells able to prevent autoimmunity. In some embodiments, immune cells can include, but are not limited to, αβT cells, γδT cells, Treg cells, NK cells, NK T cells, macrophages, or neutrophils. In some embodiments, the cells are pluripotent stem cells (embryonic stem cells and induced pluripotent stem cells) and pluripotent stem cell-derived immune cells, such as but not limited to natural killer cells, macrophages, and lymphoid cells, especially T cells. The examples of suitable processes for generating the immune cells (such as T cells, NK cells and macrophages) from the pluripotent stem cells are described (Lee, 2019, “When CAR Meets Stem Cells”, Int J Mol Sci 20).

In some embodiments, the stem cell or immune cell comprising exogenous mitochondria is an engineered cell, e.g., engineered to express a chimeric antigen receptor (CAR), such as any of the CAR formats described herein. A CAR typically includes an antigen-binding moiety (e.g., an antigen-binding domain or antigen-binding fragment thereof), a transmembrane component, and a primary cytoplasmic signaling sequence selected to activate the immune cell in response to the antigen-binding moiety binding its cognate ligand. In some embodiments, the basic components of a chimeric antigen receptor (CAR) include the following: (1) The variable heavy (V_{H}) and light (V_{L}) chains for a tumor-specific monoclonal antibody are fused in-frame with the CD3ζ-chain from the T cell receptor complex. (2) The V_{H} and V_{L} are generally connected together using a flexible glycine-serine linker, and then attached to the transmembrane domain by a spacer (e.g., CD8a stalk or C_{H}2-C_{H}3 constant domains) to extend the scFv away from the cell surface so that it can readily interact with tumor antigens. In some embodiments, the engineered immune cell comprising exogenous mitochondria is a CAR-stem cell, CAR-T cell, CAR-NK cell, CAR-NK T cell, CAR-macrophage, or CAR-neutrophil.

In some embodiments the human stem cell or immune cell comprises exogenous mitochondria, wherein the exogenous mitochondria are present in the human stem cells or in the human immune cells in an amount effective to enhance immune cell survival, immune cell basal OCR, immune cell metabolic activity, immune cell expansion or a combination thereof.

In some embodiments, the enhancement of the cell basal or maximal OCR of the immune cell comprising exogenous mitochondria is in the range (expressed in folds) of between 1.1-fold to 100-fold, such as 1.1-fold to 100-fold, such as 1.1 to 99, 1.1 to 90, 1.1 to 80, 1.1 to 70, 1.1 to 60, 1.1 to 50, 1.1 to 40, 1.1 to 30, 1.1 to 20, 1.1 to 10, 1.1 to 5, 1.1 to 2, 1.1 to 1.8, 1.1 to 1.5, 1.2 to 99, 1.2 to 90, 1.2 to 80, 1.2 to 70, 1.2 to 60, 1.2 to 50, 1.2 to 20, 1.2 to 10, 1.2 to 5, 1.2 to 2.5, 1.3 to 90, 1.3 to 80, 1.3 to 70, 1.3 to 50, 1.3 to 40, 1.3 to 30, 1.3 to 20, 1.3 to 10, 1.3 to 5, 1.3 to 1.5, 1.4 to 100, 1.4 to 95, 1.4 to 90, 1.4 to 80, 1.4 to 70, 1.4 to 60, 1.4 to 50, 1.4 to 30, 1.4 to 25, 1.4 to 20, 1.4 to 10, 1.4 to 5, 1.4 to 3, 1.4 to 2.5, 1.5 to 99, 1.5 to 95, 1.5 to 90, 1.5 to 80, 1.5 to 70, 1.5 to 60, 1.5 to 50, 1.5 to 50, 1.5 to 40, 1.5 to 30, 1.5 to 20, 1.5 to 10, 1.5 to 5, 1.5 to 2.5, 2 to 99, 2 to 90, 2 to 80, 2 to 70, 2 to 60, 2 to 50, 2 to 40, 2 to 35, 2 to 30, 2 to 20, 2 to 10, 2 to 5, 2 to 4, 2 to 2.5, 3 to 99, 3 to 90, 3 to 80, 3 to 70, 3 to 60, 3 to 50, 3 to 40, 3 to 30, 3 to 25, 3 to 20, 3 to 10, 4 to 99, 4 to 80, 4 to 70, 4 to 60, 4 to 50, 4 to 55, 4 to 25, 4 to 20, 4 to 15, 4 to 10, 5 to 100, 5 to 80, 5 to 50, 5 to 30, 5 to 20, 5 to 10, 5.5 to 9, 5.5 to 7, 10 to 90, 10 to 50, 10 to 20, 20 to 100, 20 to 50, 25 to 40, 20 to 35, 30 to 100, 30 to 50, 40 to 100, 40 to 70, 40 to 60, 40 to 50, 50 to 100, 50 to 90, 50 to 80, 50 to 70, 55 to 65, 60 to 80, 75 to 90, 75 to 100, 80 to 90, 80 to 85, 85 to 100.

In some embodiments, the enhancement of the cell expansion of the immune cell comprising exogenous mitochondria is in a the range (expressed in folds) of between 1.1 to 20 fold, such as: 1.1 to 1.5, 1.1 to 2.0, 1.1 to 3.0, 1.1 to 5.0, 1.1 to 10, 1.1 to 15, 1.1 to 20, 1.3 to 1.5, 1.3 to 2.0, 1.3 to 3.0, 1.3 to 5.0, 1.3 to 10, 1.3 to 15, 1.3 to 20, 1.5 to 2.0, 1.5 to 3.0, 1.5 to 5.0, 1.5 to 10, 1.5 to 15, 1.5 to 20, 2.0 to 3.0, 2.0 to 5.0, 2.0 to 10, 2.0 to 15, 2.0 to 20, 3.0 to 5.0, 3.0 to 10, 3.0 to 15, 3.0 to 20, 4.0 to 5.0, 4.0 to 10, 4.0 to 15, 4.0 to 20, 5.0 to 10, 5.0 to 15, 5.0 to 20, 6.0 to 10, 6.0 to 15, 6.0 to 20, 7.0 to 10, 7.0 to 15, 7.0 to 20, 8.0 to 10, 8.0 to 15, 8.0 to 20, 9.0 to 10, 9.0 to 15, 9.0 to 20, 10 to 15, 10 to 20, 11 to 15, 11 to 20, 12 to 15, 12 to 20, 13 to 15, 13 to 20, 14 to 15, 14 to 20, 15 to 20, 16 to 20, 17 to 20, 18 to 20, 19 to 20.

In some embodiments, the enhancement of the cell metabolic activity of the immune cell comprising exogenous mitochondria is in a the range is in the range (expressed in folds) of between 1.1-fold to 100-fold, such as 1.1 to 99, 1.1 to 90, 1.1 to 80, 1.1 to 70, 1.1 to 60, 1.1 to 50, 1.1 to 40, 1.1 to 30, 1.1 to 20, 1.1 to 10, 1.1 to 5, 1.1 to 2, 1.1 to 1.8, 1.1 to 1.5, 1.2 to 99, 1.2 to 90, 1.2 to 80, 1.2 to 70, 1.2 to 60, 1.2 to 50, 1.2 to 20, 1.2 to 10, 1.2 to 5, 1.2 to 2.5, 1.3 to 90, 1.3 to 80, 1.3 to 70, 1.3 to 50, 1.3 to 40, 1.3 to 30, 1.3 to 20, 1.3 to 10, 1.3 to 5, 1.3 to 1.5, 1.4 to 100, 1.4 to 95, 1.4 to 90, 1.4 to 80, 1.4 to 70, 1.4 to 60, 1.4 to 50, 1.4 to 30, 1.4 to 25, 1.4 to 20, 1.4 to 10, 1.4 to 5, 1.4 to 3, 1.4 to 2.5, 1.5 to 99, 1.5 to 95, 1.5 to 90, 1.5 to 80, 1.5 to 70, 1.5 to 60, 1.5 to 50, 1.5 to 50, 1.5 to 40, 1.5 to 30, 1.5 to 20, 1.5 to 10, 1.5 to 5, 1.5 to 2.5, 2 to 99, 2 to 90, 2 to 80, 2 to 70, 2 to 60, 2 to 50, 2 to 40, 2 to 35, 2 to 30, 2 to 20, 2 to 10, 2 to 5, 2 to 4, 2 to 2.5, 3 to 99, 3 to 90, 3 to 80, 3 to 70, 3 to 60, 3 to 50, 3 to 40, 3 to 30, 3 to 25, 3 to 20, 3 to 10, 4 to 99, 4 to 80, 4 to 70, 4 to 60, 4 to 50, 4 to 55, 4 to 25, 4 to 20, 4 to 15, 4 to 10, 5 to 100, 5 to 80, 5 to 50, 5 to 30, 5 to 20, 5 to 10, 5.5 to 9, 5.5 to 7, 10 to 90, 10 to 50, 10 to 20, 20 to 100, 20 to 50, 25 to 40, 20 to 35, 30 to 100, 30 to 50, 40 to 100, 40 to 70, 40 to 60, 40 to 50, 50 to 100, 50 to 90, 50 to 80, 50 to 70, 55 to 65, 60 to 80, 75 to 90, 75 to 100, 80 to 90, 80 to 85, 85 to 100.

In some embodiments, the enhancement of the cell survival by reducing the exhaustion of the immune cell comprising exogenous mitochondria is such that the reduction of the cell exhaustion is in the range of 0.5% to 100%, such as: 0.5% to 1.5%, 0.5% to 2.0%, 0.5% to 3.0%, 0.5% to 5.0%, 0.5% to 10%, 0.5% to 15%, 0.5% to 20%, 0.5% to 30%, 0.5% to 40%, 0.5% to 50%, 0.5% to 60%, 0.5% to 70%, 0.5% to 80%, 0.5% to 90%, 0.5% to 100%, 1.5% to 2.0%, 1.5% to 3.0%, 1.5% to 5.0%, 1.5% to 10%, 1.5% to 15%, 1.5% to 20%, 1.5% to 30%, 1.5% to 40%, 1.5% to 50%, 1.5% to 60%, 1.5% to 70%, 1.5% to 80%, 1.5% to 90%, 1.5% to 100%, 2.0% to 3.0%, 2.0% to 5.0%, 2.0% to 10%, 2.0% to 15%, 2.0% to 20%, 2.0% to 30%, 2.0% to 40%, 2.0% to 50%, 2.0% to 60%, 2.0% to 70%, 2.0% to 80%, 2.0% to 90%, 2.0% to 100%, 5.0% to 10%, 5.0% to 15%, 5.0% to 20%, 5.0% to 30%, 5.0% to 40%, 5.0% to 50%, 5.0% to 60%, 5.0% to 70%, 5.0% to 80%, 5.0% to 90%, 5.0% to 100%, 10% to 15%, 10% to 20%, 10% to 30%, 10% to 40%, 10% to 50%, 10% to 60%, 10% to 70%, 10% to 80%, 10% to 90%, 10% to 100%, 20% to 30%, 20% to 40%, 20% to 50%, 20% to 60%, 20% to 70%, 20% to 80%, 20% to 90%, 20% to 100%, 30% to 40%, 30% to 50%, 30% to 60%, 30% to 70%, 30% to 80%, 30% to 90%, 30% to 100%, 40% to 50%, 40% to 60%, 40% to 70%, 40% to 80%, 40% to 90%, 40% to 100%, 50% to 60%, 50% to 70%, 50% to 80%, 50% to 90%, 50% to 100%, 60% to 70%, 60% to 80%, 60% to 90%, 60% to 100%, 70% to 80%, 70% to 90%, 70% to 100%, 80% to 90%, 80% to 100%, 90% to 100%.

T cells

In one embodiment, the immune cell comprising or enhanced by exogenous mitochondria is a T cell (also referred to as T lymphocytes), which belongs to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocytes types such as B cells and Natural Killer (NK) cells by the presence of cell surface proteins known as T cell receptors (TCR) that recognize antigens presented on the surface of cells. During a typical immune response, binding of these antigens to the T cell receptor, in the context of MHC antigen presentation, initiates intracellular changes leading to T cell activation.

T cells are divided into two groups by T cell receptors (TCRs), αβT cells and γδT cells. αβT cells, with TCR2, mainly mediate cell immunity and immune-regulation while γδT cells, with TCR1, play important functions in wound healing, removing distressed or transformed epithelial cells and subduing excessive inflammation besides maintaining immune homeostasis in the local microenvironment. αβT cells and γδT cells play different roles in autoimmune diseases, tumors and vascular diseases. αβT cells consist of 65-75% of peripheral blood mononuclear cells (PBMC) while γδT cells account for less than 10%. They express different surface markers of CD4 and CD8, e.g., 60% αβT cells are CD4 positive, 30% CD8 positive, and both positive less than 1% in αβT cells.

The term “activated T cells” as used herein, refers to T cells that have been stimulated to produce an immune response (e.g., clonal expansion of activated T cells) by recognition of an antigenic determinant, such as, for example, presented in the context of a Class I or Class II major histocompatibility (MHC) marker. T cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T cells (e.g., cells positive for CD3, CD4, or CD8 expression are referred to as CD3⁺, CD4⁺ or CD8⁺). CD3 and CD4 proteins are cell surface receptors or co-receptors that may be directly and/or indirectly involved in signal transduction in T cells.

In some embodiments, the immune cell comprising and/or enhanced by exogenous mitochondria comprises a CAR-T cell population. In some embodiments, the CAR-T cell population is selected, or enriched, or purified, to comprise at least 20%, 30%, 40%, 50%, 55%, 60%, 65%, 70%, 75%, 80%, 85%, 90%, 95%, 96%, 97%, 98% or 99%, for example, of a cell type that expresses a certain marker, receptor, or cell surface glycoprotein, such as, for example, CD8, CD4, CD3, CD34.

In some embodiments, the CAR-T cell population include CD4⁺ and CD8⁺ T cells. In some embodiments the CAR-T cell population is enriched to comprise at least 20%, 30%, 40%, 50%, 60%, 70%, 75%, 80%, 85%, 90%, 95%, 96%, 97%, 98% or 99% CD8⁺ T cells. In some embodiments the CAR-T cell population is enriched to comprise at least 80% CD8⁺ T cells. In some embodiments the CAR-T cell population is enriched to comprise at least 90% CD8⁺ T cells. Thus, in some embodiments, there are more genetically modified CD8⁺ T cells than genetically modified CD4⁺ T cells in the composition i.e., the ratio of CD4⁺ cells to CD8⁺ cells is less than 1, e.g., less than 0.9, less than 0.8, less than 0.7, less than 0.6, or less than 0.5.

Enriched Immune Cell Populations

In some embodiments, enriched cell populations comprising or enhanced by exogenous mitochondria are provided, where the enriched cell population has been selected to comprise specified ratios or percentages of one or more cell type. By “cell population” or “modified cell population” is meant a group of cells, such as more than two cells. The cell population may be homogenous, comprising the same type of cell, or each comprising the same marker, or it may be heterogeneous. In some examples, the cell population is derived from a sample obtained from a subject and comprises cells prepared from, for example, bone marrow, umbilical cord blood, peripheral blood, or any tissue. In some examples, the cell population has been contacted with a nucleic acid, wherein the nucleic acid comprises a heterologous polynucleotide, such as, for example, a polynucleotide that encodes a chimeric antigen receptor, an inducible chimeric pro-apoptotic polypeptide, or a costimulatory polypeptide, such as, for example, a chimeric myeloid differentiation primary response 88 (MyD88) or truncated MyD88 and CD40 polypeptide. In some examples, the cell population and modified cell population are progeny of the original cells that have been contacted with the nucleic acid that comprises the heterologous polynucleotide. A cell population may be selected, or enriched, or purified, to comprise at least 20%, 30%, 40%, 50%, 55%, 60%, 65%, 70%, 75%, 80%, 85%, 90%, 95%, 96%, 97%, 98% or 99%, for example, of a cell type that expresses a certain marker, receptor, or cell surface glycoprotein, such as, for example, CD8, CD4, CD3, CD34. Without intending to be limited to any theory, in some embodiments, enriching the T cell populations to obtain increased ratios of CD8⁺ to CD4⁺ T cells may reduce the level of CAR-T cell associated cytokine-release syndrome and neurotoxicity.

The efficacy of chimeric antigen receptor-modified immune cells (e.g., a CAR T cell) is typically dependent on their in vivo expansion following adoptive transfer. Additional genetic augmentations to improve CAR-T expansion may improve therapeutic efficacy but may risk increasing CAR-T toxicity. CAR-T cells, CAR-T cells that express costimulatory polypeptides, and CAR-T cells that express MyD88, or MyD88-CD40 chimeric proteins either constitutively or under the control of an inducible multimerizing region, are effective at eliminating tumors but may induce acute cytokine-related toxicity. The potential for cytotoxicity may reduce the dosage of CAR-T cells that may be administered to a subject. The EXAMPLES section shows that mitochondria transplantation may lead to the CAR-T cells with more benign safety profile. Accordingly, in some embodiments, provided for herein are immune cells comprising or enhanced by exogenous mitochondria, such as CAR T cells, that are less toxic in immunotherapy applications than immune cells not comprising exogenous mitochondria.

Collecting T Lymphocytes from Patient's Blood and Enrichment of T Cells

T cells, such as T cells enhanced by exogenous mitochondria and/or engineered to express a CAR, can be derived from any healthy donor. The donor will generally be an adult (at least 18 years old) but children are also suitable as T cell donors (Styczynski, 2018, “Young child as a donor of cells for transplantation andlymphocyte based therapies”, Transfus Apher Sci 57:323-30). An example of a suitable process for obtaining T cells from a donor is described in (Di Stasi et al., 2011, “Inducible apoptosis as a safety switch for adoptive cell therapy”, N Engl J Med 365:1673-83). In general, T cells are obtained from a donor, subjected to genetic modification and selection, and can then be administered to recipient subjects. A useful source of T cells is the donor's peripheral blood. Peripheral blood samples will generally be subjected to leukapheresis to provide a sample enriched for white blood cells. This enriched sample (also known as a “leukopak”) can be composed of a variety of blood cells including monocytes, lymphocytes, platelets, plasma, and red cells. Elimination of contaminants, like red blood cells, platelets, monocytes, and tumor cells, requires a multi-pronged approach generally required using methods known in the art. A leukopak typically contains a higher concentration of cells as compared to venipuncture or buffy coat products.

Patients with relapsed cancer may have low T-cell counts, thus making it difficult to collect sufficient autologous T cells. This issue can be overcome by methods known in the art, such as by using allogeneic T lymphocytes collected from healthy donors.

The selection, enrichment, or purification of a cell type in the modified cell population may be achieved by any suitable method. In some embodiments, the proportions of CD8⁺ and CD4⁺ T cells may be determined by flow cytometry. In some examples, a MACs column may be used. In some examples, the modified cell population is frozen and defrosted before administration to the subject, and the viable cells are tested for the percentage or ratio of a certain cell type before administration to the subject. T cells are separated into purified CD4⁺ and CD8⁺ T cells by magnetic selection (MACS columns), following transduction or transfection. The composition may include CD4⁺ and CD8⁺ T cells, and ideally, the population of genetically modified CD3⁺ T cells within the composition includes both CD4⁺ and CD8⁺ cells. Whereas the ratio of CD4⁺ cells to CD8⁺ cells in a leukopak is typically above 2, in some embodiments the ratio of genetically modified CD4⁺ cells to genetically modified CD8⁺ cells in a composition of the invention is less than 2, e.g., less than 1.5. In some embodiments, there are more genetically modified CD8⁺ T cells than genetically modified CD4⁺ T cells in the composition, i.e., the ratio of CD4⁺ cells to CD8⁺ cells is less than 1 e.g. less than 0.9, less than 0.8, less than 0.7, less than 0.6, or less than 0.5. Thus, the overall procedure starting from donor cells and producing genetically modified T cells is designed to enrich for CD8⁺ cells T cells relative to CD4⁺ T cells. In some embodiments, 60% or more of the genetically modified T cells are CD8⁺ T cells, and in some embodiments, 65% or more of the genetically modified T cells are CD8⁺ T cells. Within the population of genetically modified CD3⁺ T cells, in some embodiments, the percent of CD8⁺ T cells is between 55-75%, for example, from 55%-65%, from 55%-70%, from 56-71%, from 63-73%, from 60-70%, from 59%-74%, from 65-71% or from 65-75%. In some embodiments, a cell population is provided that is selected, or enriched, or purified, to comprise a ratio of one cell type to another, such as, for example, a ratio of CD8⁺ to CD4⁺ T cells of, for example, 3:2, 7:3, 4:1, 9:1, 19:1, or 39:1 or more. In some embodiments, the modified cell population is selected, or enriched, or purified, to comprise at least 20%, 30%, 40%, 50%, 55%, 60%, 65%, 70%, 75%, 80%, 85%, 90%, 95, 96, 97, 98, or 99%, CD8⁺ T cells. In some embodiments, the ratio of CD8⁺ to CD4⁺ T cells is 4-to-1, or 9-to-1 or greater.

In some embodiments, for a population of genetically modified CD3⁺ T cells comprising a costimulatory polypeptide as described herein, the percent of CD8⁺ T cells is between 55-75%, for example, from 55-65%, from 55-70%, from 56-71%, from 59-74%, from 63-73%, from 60-70%, from 60-75%, from 65-75%, or from 65-71%. In some embodiments, the ratio of CD8⁺ to CD4⁺ T cells is 3:2, 7:3, 4:1, 9:1, 19:1, or 39:1 or more. In some embodiments, the modified cell population comprising a costimulatory polypeptide is selected, or enriched, or purified, to comprise at least 20%, 30%, 40%, 50%, 60%, 70%, 75%, 80%, 85%, 90%, 95, 96, 97, 98, or 99%, CD8⁺ T cells. In some embodiments, the ratio of CD8⁺ to CD4⁺ T cells is 4-to-1, or 9-to-1 or greater. The costimulatory polypeptide can comprise one or more costimulatory signaling regions such as CD27, ICOS, RANK, TRANCE, CD28, 4-1BB, OX40, DAP10, MyD88, or CD40. The costimulatory polypeptide can comprise one or more costimulatory signaling regions that activate the signaling pathways activated by CD27, ICOS, RANK, TRANCE, CD28, 4-1BB, OX40, DAP10, MyD88, or CD40. The costimulatory polypeptide can be inducible or constitutively activated.

In some embodiments, the invention provides compositions and methods comprising a CAR-T cell population comprising an inducible pro-apoptotic polypeptide where at least 80%, 85%, 90%, 95, 96, 97, 98, or 99%, are CD8⁺ T cells. In some embodiments, the modified cell population comprising an inducible pro-apoptotic polypeptide is at least 80% CD8⁺ T cells. In some embodiments, the modified cell population is at least comprising an inducible pro-apoptotic polypeptide 90% CD8⁺ T cells.

In some embodiments, the invention provides compositions and methods comprising a CAR-T cell population comprising a costimulatory polypeptide and an inducible pro-apoptotic polypeptide where at least 80%, 85%, 90%, 95, 96, 97, 98, or 99%, are CD8⁺ T cells. In some embodiments, the modified cell population comprising a costimulatory polypeptide and an inducible pro-apoptotic polypeptide is at least 80% CD8⁺ T cells. In some embodiments, the modified cell population comprising a costimulatory polypeptide and an inducible pro-apoptotic polypeptide is at least 90% CD8⁺ T cells.

According to the present disclosure, mitochondria preparations comprising e.g., autologous mitochondria, allogeneic mitochondria, xenogeneic mitochondria, encapsulated mitochondria or autogenous mitochondria with appropriate genetic modification may be delivered to enriched T cells before, concurrently with, or after genetic modification (e.g., introduction of the CAR gene) is performed.

Mitochondria

The present invention is based, at least in part, on the discovery that isolated mitochondria can be delivered to (also referred to as transplanted into) cultured cells or a patient's tissue by adding them to a cell culture or by injecting them into the patient's tissue or blood vessels leading to the tissue, respectively (Cowan et al., 2017, “Transit and integration of extracellular mitochondria in human heart cells”, Sci Rep 7:17450; McCully et al., 2017, “Mitochondrial transplantation: From animal models to clinical use in humans”, Mitochondrion 34:127-34).

Mitochondria can be delivered ex vivo to cells of interest. Cells of interest include, but are not limited to, any of the immune cells described herein, stem cells and/or cells differentiated therefrom, cultured cells, previously engineered immune cells (e.g., CAR T cells), or cells to be further engineered (e.g., to express a CAR or artificial TCR) and/or cultured (e.g., differentiated, activated, treated, or incubated). Mitochondria can be delivered ex vivo by liposome-mediated transfer using the synthetic liposomes, such as Lipofectin® (Shi et al., 2008. “Mitochondria transfer into fibroblasts: liposome-mediated transfer of labeled mitochondria into cultured cells”, Ethn. Dis. 18: S1-43). Mitochondria can be delivered ex vivo through co-incubation (i.e., co-culturing) of the cells, such as any of the immune cells described herein, with mitochondria over the period of 2-24 hours (Masuzawa et al., 2013, “Transplantation of autologously derived mitochondria protects the heart from ischemia-reperfusion injury”, Am J Physiol Heart Circ Physiol 304:H966-82). Without wishing to be bound by theory, transplanted mitochondria are internalized by an actin dependent pathway. Mitochondrial internalization, such as previously demonstrated in cardiomyocytes, can occur following a 1-hour co-incubation (Pacak et al., 2015, “Actin-dependent mitochondrial internalization in cardiomyocytes: evidence for rescue of mitochondrial function”, Biol Open 4:622-6).

Mitochondria can also be delivered into an organ or tissue by direct injection into the targeted area, or by delivery through the organ- or tissue-specific vasculature, such as the coronary artery of the subject, the pulmonary artery of the subject, the hepatic portal vein of the subject, the greater pancreatic artery of the subject, the renal artery of the subject, or the prostate artery of the subject. In the latter case, mitochondria are retained in the downstream organ or tissue. For example, when administered through the coronary arteries, mitochondria are almost exclusively delivered to the heart (Shin et al., 2019, “Myocardial Protection by Intracoronary Delivery of Mitochondria: Safety and Efficacy in the Ischemic Myocardium”, JACC: Basic to Translational Science Vol. 4, No. 8, 2019), while the mitochondria may be delivered into the lung through the pulmonary artery, or into the kidneys by delivery through the renal arteries. The direct injection of mitochondria allows for focal concentration of the injected mitochondria. The number of mitochondria used for injection may vary, depending on the size of the targeted organ or tissue as well as the intended use. The mitochondria may be suspended in respiration buffer and injected at various sites using e.g. a tuberculin syringe with a 28-32 gauge needle (Emani et al., 2017, “Autologous mitochondrial transplantation for dysfunction after ischemia-reperfusion injury”, J Thorac Cardiovasc Surg 154:286-9; McCully et al., 2017, “Mitochondrial transplantation: From animal models to clinical use in humans”, Mitochondrion 34:127-34).

Mitochondrial transplantation in vivo can be performed using either single or serial injections of either autologous or heterologous mitochondria, with no direct or indirect, acute or chronic alloreactivity, allorecognition, or damage-associated molecular pattern molecules (Ramirez-Barbieri et al., 2019, “Alloreactivity and allorecognition of syngeneic and allogeneic mitochondria”, Mitochondrion 46:103-15).

Without wishing to be bound by theory, viable, respiration competent mitochondria are taken up by both ischemic and non-ischemic tissue by endocytosis (Cowan et al., 2016, “Intracoronary Delivery of Mitochondria to the Ischemic Heart for Cardioprotection”, PLoS One 11:e0160889; Kesner et al., 2016, “Characteristics of Mitochondrial Transformation into Human Cells”, Sci Rep 6:26057; Cowan et al., 2017, “Transit and integration of extracellular mitochondria in human heart cells”, Sci Rep 7:17450).

Skilled practitioners can locally and/or generally distribute mitochondria to tissues and/or cells of a patient for a variety of purposes, using relatively simple medical procedures. Compared to some traditional therapeutic regimens that involve nanoparticles, it is further noted that mitochondria are not toxic and do not cause any substantial adverse immune or auto-immune response.

While not intending to be bound by any theory, it is believed that infused mitochondria extravasate through the capillary wall by first adhering to the endothelium. After they are injected or infused into an artery, mitochondria can cross the endothelium of the blood vessels and be taken up by tissue cells through an endosomal actin-dependent internalization process.

Mitochondrial transplantation in vivo can include co-administration of any of the cells of interest described herein together with the exogenous mitochondria provided herein. In some embodiments, exogenous mitochondria and cells of interest are co-administered to promote or enhance the desired therapeutic effect of the cells of interest to treat a disease in a patient. Cells of interest include, but are not limited to, any of the immune cells described herein, stem cells and/or cells differentiated therefrom, cultured cells, previously engineered immune cells (e.g., CAR T cells), or cells to be further engineered (e.g., to express a CAR or artificial TCR). In embodiments where exogenous mitochondria and the cells of interest are included in different pharmaceutical compositions, administration of the exogenous mitochondria can occur prior to, simultaneously with, and/or following, administration of the cells of interest. In some aspects, administration of exogenous mitochondria and cells of interest occur within about one month of each other. In some aspects, administration of exogenous mitochondria and cells of interest occur within about one week of each other. In some aspects, administration of exogenous mitochondria and the cells of interest occur within about five, four, three or two days of each other. In some aspects, administration of exogenous mitochondria and the cells of interest occur within about one day of each other. In some aspects, administration of exogenous mitochondria and cells of interest occur within about twelve hours of each other. In some aspects, administration of exogenous mitochondria and cells of interest occur within about six hours of each other. In some aspects, administration of exogenous mitochondria and cells of interest occur within about three hours of each other. In some aspects, administration of exogenous mitochondria and cells of interest occur within about two hours of each other. In some aspects, administration of exogenous mitochondria and cells of interest occur within about one hour of each other. In some aspects, administration of exogenous mitochondria and cells of interest occur within about thirty minutes of each other. In some aspects, administration of exogenous mitochondria and cells of interest occur within about fifteen minutes of each other. In some aspects, administration of exogenous mitochondria and the cells of interest occur within minutes of each other. In some aspects, co-administration of exogenous mitochondria and cells of interest include repeated administration of exogenous mitochondria and/or cells of interest.

Isolating Mitochondria

Mitochondria for use in the presently described methods can be isolated or provided from any source, e.g., isolated from cultured cells or tissues. Exemplary cells include, but are not limited to, muscle tissue cells, cardiac fibroblasts, HeLa cells, prostate cancer cells, yeast, among others, and any mixture thereof. Exemplary tissues include, but are not limited to, liver tissue, skeletal muscle, heart, brain, and adipose tissue. Mitochondria can be isolated from cells or tissues (e.g., biopsy material) of an autogenous source, an allogeneic source, and/or a xenogeneic source. In some instances, mitochondria are isolated from cells with a genetic modification, e.g., cells with modified mtDNA or modified nuclear DNA.

Mitochondria can be isolated from cells or tissues by any means known to those of skill in the art. In one example, tissue samples or cell samples are collected and then homogenized. Following homogenization, mitochondria are isolated by repetitive centrifugation (Kesner et al., 2016, “Characteristics of Mitochondrial Transformation into Human Cells”, Sci Rep 6:26057). Alternatively, the cell homogenate can be filtered through nylon mesh filters. Typical methods of isolating mitochondria are described, for example, in McCully J D, Cowan D B, Pacak C A, Toumpoulis I K, Dayalan H and Levitsky S, “Injection of isolated mitochondria during early reperfusion for cardioprotection”, Am J Physiol 296, H94-H105. PMC2637784 (2009); Frezza, C., Cipolat, S., & Scorrano, L, “Organelle isolation: functional mitochondriafrom mouse liver, muscle and cultured filroblasts”, Nature protocols, 2(2), 287-295 (2007); and a PCT application entitled “Products and Methods to Isolate Mitochondria” (PCT/US2015/035584; WO 2015192020); each of which is incorporated by reference.

Mitochondria, such as those used in therapy or included in a pharmaceutical composition, can be isolated from cells or tissues of an autogenous source, an allogeneic source, or a xenogeneic source. In some instances, mitochondria are collected from cultured cells or tissues of a subject, and these mitochondria are administered back to the same subject (autologous). In some other cases, mitochondria are collected from cultured cells (e.g., human cardiac fibroblasts) or tissues of a second subject, and these mitochondria are administered to a first subject (allogeneic). In some cases, mitochondria are collected from cultured cells or tissues from a different species (e.g., mice, swine, and yeast) (xenogeneic).

In certain embodiments of methods described herein, the mitochondria can have different sources, e.g., the exogenous mitochondria can be autologous, autogeneic, allogeneic, or xenogeneic. In certain embodiments the mitochondria have been freshly isolated (within 120 min after taking the tissue biopsy samples). In some embodiments the mitochondria have been isolated and subsequently stored until use. In certain embodiments, the autogeneic mitochondria can have exogenous mtDNA. In some embodiments, the mitochondria are from a subject's first-degree relative. In some embodiments, the mitochondria have been encapsulated.

In some embodiments, the described methods include the step of collecting the isolated mitochondria from cells prior to administration. The isolated mitochondria can be transplanted into cells of interest, e.g., any of the immune effector cells described herein, or administered to the subject in conjunction with the treatment with cells of interest.

In the present disclosure, exogenous mitochondria are transplanted into immune cells to boost their survival, activity or a combination thereof with the result of an Enhanced Adoptive Cell Transfer (EACT). Alternatively, or additionally, mitochondria are introduced into the patient in conjunction with delivering an ACT or EACT with the result of increasing the efficacy or safety of an ACT or EACT.

Engineering Expression Constructs

In some embodiments, the immune cell comprising or enhanced by exogenous mitochondria is engineered, such as engineered to express a CAR. Expression constructs that express the present chimeric antigen receptors, chimeric signaling polypeptides, and inducible safety switches are provided herein.

As used herein, the term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

In certain examples, a polynucleotide coding for the chimeric antigen receptor, is included in the same vector, such as, for example, a viral or plasmid vector, as a polynucleotide coding for a second polypeptide. This second polypeptide may be, for example, a chimeric signaling polypeptide, an inducible caspase polypeptide, as discussed herein, or a marker polypeptide. In these examples, the construct may be designed with one promoter operably linked to a nucleic acid comprising a polynucleotide coding for the two polypeptides, linked by a 2A polypeptide. In this example, the first and second polypeptides are separated during translation, resulting in two polypeptides, or, in examples including a leaky 2A, either one, or two polypeptides. In other examples, the two polypeptides may be expressed separately from the same vector, where each nucleic acid comprising a polynucleotide coding for one of the polypeptides is operably linked to a separate promoter. In yet other examples, one promoter may be operably linked to the two polynucleotides, directing the production of two separate RNA transcripts, and thus two polypeptides; in one example, the promoter may be bi-directional, and the coding regions may be in opposite directions 5′-3′. Therefore, the expression constructs discussed herein may comprise at least one, or at least two promoters.

In some embodiments, a nucleic acid construct, e.g., any of the chimeric antigen receptors described herein, is contained within a viral vector. In certain embodiments, the viral vector is a retroviral vector. In certain embodiments, the viral vector is an adenoviral vector or a lentiviral vector. It is understood that in some embodiments, a cell is contacted with the viral vector ex vivo, and in some embodiments, the cell is contacted with the viral vector in vivo. Thus, an expression construct may be inserted into a vector, for example a viral vector or plasmid. The steps of the methods provided may be performed using any suitable method; these methods include, without limitation, methods of transducing, transforming, or otherwise providing nucleic acid to the cell, described herein.

As used herein, the term “gene” is defined as a functional protein-, polypeptide-, or peptide encoding unit. As will be understood, this functional term includes genomic sequences, cDNA sequences, and smaller engineered gene segments that express, or are adapted to express, proteins, polypeptides, domains, peptides, fusion proteins and/or mutants.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, and at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity. As used herein, the term “promoter” is defined as a DNA sequence recognized by the synthetic machinery of the cell, or introduced synthetic machinery, required to initiate the specific transcription of a gene. In some embodiments, the promoter is a developmentally regulated promoter. As used herein, the term “under transcriptional control”, “operably linked”, or “operatively linked” is defined as the promoter is in the correct location and orientation in relation to the nucleic acid to control RNA polymerase initiation and expression of the gene. In some examples, one or more polypeptides are said to be “operatively linked”. In general, the term “operably linked” is meant to indicate that the promoter sequence is functionally linked to a second sequence, wherein the promoter sequence initiates and mediates transcription of the DNA corresponding to the second sequence.

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Such a promoter might include either a human or viral promoter. Promoters may be selected that are appropriate for the vector used to express the CARs and other polypeptides provided herein.

In various embodiments, where, for example, the expression vector is a retrovirus, e.g., a lentivirus, an example of an appropriate promoter is the Murine Moloney leukemia virus (MMLV) promoter. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Lentiviral vectors can include a self-inactivating lentiviral vector as provided in Milone et al., Mol. Ther. 17(8): 1453-1464 (2009). Other examples of lentiviral vectors that may be used in the clinic include but are not limited to the LENTIVECTOR™ gene delivery technology from Oxford BioMedica, the LENTIMAX™ vector system from Lentigen, and the like. Nonclinical types of lentiviral vectors are also available and would be known to one skilled in the art.

In other embodiments, the promoter may be, for example, the cytomegalovirus (CMV) immediate early gene promoter, the simian virus 40 (SV40) early promoter, the Rous sarcoma virus (RSV) long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters, which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Promoters, and other regulatory elements, are selected such that they are functional in the desired cells or tissue. In addition, this list of promoters should not be construed to be exhaustive or limiting; other promoters that are used in conjunction with the promoters and methods disclosed herein.

The nucleic acids discussed herein may comprise one or more polynucleotides. In some embodiments, one or more polynucleotides may be described as being positioned, or is 5′ or 3′ of another polynucleotide or positioned in 5′-to-3′ order. The reference 5′-to-3′ in these contexts is understood to refer to the direction of the coding regions of the polynucleotides in the nucleic acid, for example, where a first polynucleotide is positioned 5′ of a second polynucleotide and connected with a third polynucleotide encoding a non-cleavable linker polypeptide, the translation product would result in the polypeptide encoded by the first polynucleotide positioned at the amino-terminal end of a larger polypeptide comprising the translation products of the first, third, and second polynucleotides.

Expression constructs, such as CAR genes, can be incorporated randomly into the genome, such as through viral mediated integration, or purposely integrated into the specific sites of an immune cell genome, such as a T-cell genome, including but not limited to CCR5 and AAVS1 loci, or into the T-cell receptor a constant (TRAC) locus. Targeted integration can use gene-editing tools such as nuclease-meditated genome editing systems, including the clustered regularly interspaced short palindromic repeats (CRISPR/Cas9) system, zinc-finger nucleases (ZFNs), and transcription activator-like effector nucleases (TALENs) (Liu et al., 2019, “Building Potent Chimeric Antigen Receptor T Cells With CRISPR Genome Editing”, Front Immunol 10:456).

Costimulation

In some embodiments, the immune cell comprising or enhanced by exogenous mitochondria is an immune cell engineered to express a CAR, such as a CAR-T cell, comprising a costimulatory polypeptide. In some embodiments, the immune cell comprising or enhanced by exogenous mitochondria is a CAR-T cell comprising a costimulatory polypeptide. The CARs can be engineered to include a costimulation domain, such as those derived from the cytoplasmic portion of T cell costimulatory molecules, including, but not limited to, CD28, 4-1BB, OX40, ICOS and DAP10 (see, e.g., Carpenito et al. (2009) Proc Natl Acad Sci U.S.A. 106:3360-3365; Finney et al. (1998) J Immunol 161:2791-2797; Hombach et al. J Immunol 167:6123-6131; Maher et al. (2002) Nat Biotechnol 20:70-75; Imai et al. (2004) Leukemia 18:676-684; Wang et al. (2007) Hum Gene Ther 18:712-725; Zhao et al. (2009) J Immunol 183:5563-5574; Milone et al. (2009) Mol Ther 17: 1453-1464; Yvon et al. (2009) Clin Cancer Res 15:5852-5860), which allow CAR-T cells to receive appropriate costimulation upon engagement of the target antigen. Costimulatory molecules can include CD28 and 4-1BB (CD137), which, following tumor recognition, can initiate a signaling cascade resulting in NF-κB activation, which promotes both T cell proliferation and cell survival. Clinical trials conducted with anti-CD19 CARs having CD28 or 4-1BB signaling domains for the treatment of refractory acute lymphoblastic leukemia (ALL) have demonstrated significant T cell persistence, expansion and serial tumor killing following adoptive transfer (Kalos et al. (2011) Sci Transl Med 3:95ra73; Porter et al. (2011) N Engl J Med 365:725-733; Brentjens et al. (2013) Sci Transl Med 5: 177ra38). Third generation CAR-T cells append CD28-modified CARs with additional signaling molecules from tumor necrosis factor (TNF)-family proteins, such as OX40 and 4-1BB (Finney H M, et al. J Immunol 172: 104-13, 2004; Guedan S, et al., Blood, 2014).

Some second and third-generation CAR-T cells have been implicated in patient deaths, due to cytokine storm and tumor lysis syndrome caused by highly activated T cells. In one aspect, the invention described herein of enhancing immune cells through delivery of exogenous mitochondria relates to compositions and methods comprising CAR-T cell comprising costimulatory polypeptides for enhancing and maintaining chimeric antigen receptor expressing T cells, while reducing cytotoxic effects of CAR-T cell therapies.

The costimulatory polypeptide of the present invention can be inducible or constitutively activated. The costimulatory polypeptide can comprise one or more costimulatory signaling regions such as CD27, ICOS, RANK, TRANCE, CD28, 4-1BB, OX40, DAP10, MyD88, or CD40 or, for example, the cytoplasmic regions thereof. The costimulatory polypeptide can comprise one or more suitable costimulatory signaling regions that activate the signaling pathways activated by CD27, ICOS, RANK, TRANCE, CD28, 4-1BB, OX40, DAP10, MyD88, or CD40. Costimulatory polypeptides include any molecule or polypeptide that activates the NF-κB pathway, Akt pathway, and/or p38 pathway of tumor necrosis factor receptor (TNFR) family (i.e., CD40, RANK/TRANCE-R, OX40, 4-1BB) and CD28 family members (CD28, ICOS). More than one costimulatory polypeptide or costimulatory polypeptide cytoplasmic region may be expressed in the modified T cells discussed herein.

In some embodiments, the CAR-T cell population comprising the costimulatory polypeptide is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the CAR-T cell population comprising the costimulatory polypeptide include CD4⁺ and CD8⁺ T cells. In some embodiments the CAR-T cell population comprising the costimulatory polypeptide is enriched to comprise at least 20%, 30%, 40%, 50%, 60%, 70%, 80%, 90%, 95, or 99% CD8⁺ T cells. In some embodiments the CAR-T cell population comprising the costimulatory polypeptide is enriched to comprise at least 80% CD8⁺ T cells. In some embodiments the CAR-T cell population comprising the costimulatory polypeptide is enriched to comprise at least 90% CD8⁺ T cells. Thus, in some embodiments, there are more genetically modified CD8⁺ T cells than genetically modified CD4⁺ T cells in the composition i.e., the ratio of CD4⁺ cells to CD8⁺ cells is less than 1 e.g. less than 0.9, less than 0.8, less than 0.7, less than 0.6, or less than 0.5.

One of the principal functions of second-generation CAR T cells is the ability to produce IL-2 that supports T cell survival and growth through activation of the nuclear factor of activated T cells (NFAT) transcription factor by CD3ζ (signal 1) and NF-κB (signal 2) by CD28 or 4-1BB.

Other molecules that similarly activate NF-κB may also be paired with the CD3ζ chain within a CAR molecule. One approach employs a T cell costimulatory molecule that was originally developed as an adjuvant for a dendritic cell (DC) vaccine (Narayanan et al. (2011) J Clin Invest 121:1524-1534; Kemnade et al. (2012) Mol Ther 20(7):1462-1471). For full activation or licensing of DCs, Toll-like receptor (TLR) signaling is usually involved. In TLR signaling, the cytoplasmic TLR/IL-1 domains (referred to as TIR domains) of TLRs dimerize which leads to recruitment and association of cytosolic adaptor proteins such as, for example, the myeloid differentiation primary response protein (MyD88). MyD88 is a cytosolic adapter protein that plays a central role in the innate and adaptive immune response. This protein functions as an essential signal transducer in the interleukin-1 and Toll-like receptor (TLR) signaling pathways. These pathways regulate that activation of numerous pro-inflammatory genes. TLR signaling also upregulates expression of CD40, a member of the tumor necrosis factor receptor (TNFR) family, which interacts with CD40 ligand (CD154 or CD40L) on primed CD4⁺ T cells. CD40 is an important part of the adaptive immune response, aiding to activate APCs through engagement with its cognate CD40L, in turn polarizing a stronger CTL response. The CD40/CD154 signaling system is an important component in T cell function and B cell/T cell interactions. CD40 signaling proceeds through formation of CD40 homodimers and interactions with TNFR-associated factors (TRAFs), carried out by recruitment of TRAFs to the cytoplasmic domain of CD40, which leads to T cell activation involving several secondary signals such as the NF-κB, INK and AKT pathways.

Apart from survival and growth advantages, MyD88 or MyD88-CD40 fusion chimeric polypeptide-based stimulation may also provide additional functions to CAR-modified cells. MyD88 signaling is generally important for both Th1 and Th17 responses and acts via IL-1 to render CD4⁺ T cells refractory to regulatory T cell (Treg)-driven inhibition (see, e.g., Schenten et al. (2014) Immunity 40:78-90). In addition, CD40 signaling in CD8⁺ T cells via Ras, PI3K and protein kinase C, results in NF-κB-dependent induction of cytotoxic mediators granzyme and perforin that lyse CD4⁺CD25⁺ Treg cells (Martin et al. (2010) J Immunol 184:5510-5518). Thus, MyD88 and CD40 co-activation may render CAR-T cells resistant to the immunosuppressive effects of Treg cells, a function that could be critically important in the treatment of solid tumors and other types of cancers. One approach to costimulation of CAR-engineered cells, such as CAR-T cells, is to express a fusion protein (referred to as MC) of the signaling elements of MyD88.

In some embodiments, the inducible chimeric signaling polypeptide comprises two costimulatory polypeptide cytoplasmic signaling regions, such as, for example, 4-1BB and CD28, or one, or two or more costimulatory polypeptide cytoplasmic signaling regions selected from the group consisting of CD27, ICOS, RANK, TRANCE, CD28, 4-1BB, OX40, DAP10. In some embodiments, CAR-engineered cells, such as CAR-T cells, comprise a nucleic acid that encodes a first polynucleotide encoding the inducible chimeric signaling polypeptide and a second polynucleotide encoding the CAR. In some embodiments, the first polynucleotide is positioned 5′ of the second polynucleotide. In some embodiments, the first polynucleotide is positioned 3′ of the second polynucleotide. In some embodiments, a third polynucleotide encoding a linker polypeptide is positioned between the first and second polynucleotides. In some embodiments, the linker polypeptide is a 2A polypeptide, which may separate the polypeptides encoded by the first and second polynucleotides during, or after translation.

Vectors

In some embodiments, the population of immune cells comprising or enhanced by exogenous mitochondria (e.g., as autologous, allogeneic mitochondria, xenogeneic mitochondria, encapsulated mitochondria or autogenous mitochondria with appropriate genetic modification) comprises a CAR or artificial TCR subunit produced from a DNA, double-stranded RNA, single-stranded mRNA, or circular RNA vector. It is understood that the vectors provided herein may be modified using methods known in the art to vary the position or order of the regions, to substitute one region for another. For example, a vector comprising a polynucleotide encoding a chimeric signaling polypeptide can include a polynucleotide encoding chimeric signaling polypeptide comprising one, or two or more co-stimulatory polypeptide cytoplasmic signaling regions such as, for example, those selected from the group consisting of CD27, CD28, 4-1BB, OX40, ICOS, RANK, TRANCE, and DAP10, arranged in various orders. The polynucleotide encoding a CAR may also be modified so that the antigen-binding domain may be substituted with one having the same, or different target specificity; the transmembrane region may be substituted with a different transmembrane region; a stalk polypeptide may be added. Polynucleotides encoding marker polypeptides may be included within or separate from one of the polypeptides; polynucleotides encoding additional polypeptides coding for safety switches may be added, polynucleotides coding for linker polypeptides, or non-coding polynucleotides or spacers may be added, or the order of the polynucleotides 5′-to-3′ may be changed.

A vector can encode antigen-binding domains, e.g., as part of a CAR construct, specific for one or more target antigens, such as, for example, BCMA, CD123, CD20, CD22, CD30, CD33, EGFR, EGFRvIII, GD2, Her2, Mesothelin, MUC1, MUC16, NKG2D, NY-ESO-1, PRAME, PSCA, PSMA, ROR1, etc. The vector may also be modified with appropriate substitutions of each polypeptide region, as discussed herein.

A vector can encode co-stimulatory polypeptide cytoplasmic signaling regions, e.g., as part of a CAR construct, comprising one, or two or more co-stimulatory polypeptide cytoplasmic signaling regions such as, for example, those selected from the group consisting of CD27, CD28, 4-1BB, OX40, ICOS, RANK, TRANCE, and DAP10. Co-stimulating polypeptides may comprise, but are not limited to, the amino acid sequences provided herein, and may include functional conservative mutations, including deletions or truncations, and may comprise amino acid sequences that are 70%, 75%, 80%, 85%, 90%, 95% or 100% identical to the amino acid sequences provided herein.

A vector can encode a linker, e.g., as part of a CAR construct, such as a linker between the CAR polypeptide and the co-stimulatory polypeptide. For example, nucleic acids provided herein may comprise a costimulatory polypeptide signaling region 3′ of a polynucleotide coding for the CD3ζ portion of the CAR, where the two polynucleotides are separated by a polynucleotide coding for a linker. In some embodiments, the two polynucleotides may be separated by a polynucleotide coding for a linker polypeptide having, for example, about 5 to 20 amino acids, or, for example, about 6 to 10 amino acids.

Engineered immune cells, such as T cells (e.g., CAR T cells), of the invention may express a safety switch, also known as an inducible suicide gene or suicide switch, which can be used to eradicate the engineered immune cells in vivo if desired e.g. if graft versus host disease (GVHD) develops. In some examples, engineered immune cells that express a chimeric antigen receptor are provided to the patient that trigger an adverse event, such as on-target off-tumor toxicity. In some therapeutic instances, a patient might experience some negative symptoms during therapy using CAR-modified cells. In some cases, these therapies have led to adverse events due, in part, to non-specific attacks on healthy tissue. In some examples, the therapeutic engineered immune cells may no longer be needed, or the therapy is intended for a specified amount of time, for example, the therapeutic engineered immune cells may work to decrease the tumor cell, or tumor size, and may no longer be needed. Therefore, in some embodiments are provided nucleic acids, cells, and methods wherein the engineered immune cell also expresses a safety switch, such as an inducible caspase-9 polypeptide. Other suicide switch systems known in the art include, but are not limited to, (a) herpes simplex virus (HSV)-tk which turns the nontoxic prodrug ganciclovir (GCV) into GCV-triphosphate, leading to cell death by halting DNA replication, (b) iCasp9 can bind to the small molecule AP1903 and result in dimerization, which activates the intrinsic apoptotic pathway, and (c) Targetable surface antigen expressed in the transduced iNKT cells (e.g., CD20 and truncated EGFR), allowing eliminating the modified cells efficiently through complement/antibody-dependent cellular cytotoxicity (CDC/ADCC) after administration of the associated monoclonal antibody. If there is a need, for example, to reduce the number of engineered immune cells, an inducible ligand may be administered to the patient, thereby inducing apoptosis of the engineered immune cells. These switches respond to a trigger, such as a pharmacological agent, which is supplied when it is desired to eradicate the engineered immune cells, and which leads to cell death (e.g., by triggering necrosis or apoptosis). These agents can lead to expression of a toxic gene product, but a more rapid response can be obtained if the engineered immune cells already express a protein, which is switched into a toxic form in response to the agent.

Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually, the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus thymidine kinase (tk) are employed. Immunologic surface markers containing the extracellular, non-signaling domains or various proteins (e.g., CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, β-gal or chloramphenicol acetyltransferase (CAT). In certain embodiments, the marker protein, such as, for example, CD19 is used for selection of the cells for transfusion, such as, for example, in immunomagnetic selection. As discussed herein, a CD19 marker is distinguished from an anti-CD19 antibody, or, for example, a scFv, TCR, or other antigen recognition moiety that binds to CD19.

In certain embodiments, the marker polypeptide is linked to the inducible chimeric stimulating molecule. For example, the marker polypeptide may be linked to the inducible chimeric stimulating molecule via a polypeptide sequence, such as, for example, a cleavable 2A-like sequence.

In some embodiments, a polypeptide may be included in the polypeptide, for example, the CAR encoded by the expression vector to aid in sorting cells. In some embodiments, the expression vectors used to express the chimeric antigen receptors or chimeric stimulating molecules provided herein further comprise a polynucleotide that encodes the 16 amino acid CD34 minimal epitope. In some embodiments, such as certain embodiments provided in the examples herein, the CD34 minimal epitope is incorporated at the amino terminal position of the CD8 stalk.

Linker Polypeptides

Linker polypeptides include, for example, cleavable and non-cleavable linker polypeptides. Non-cleavable polypeptides may include, for example, any polypeptide that may be operably linked between the costimulatory polypeptide cytoplasmic signaling region and ITAM portion of the chimeric antigen receptor (e.g., CD3ζ). Linker polypeptides include those for example, consisting of about 2 to about 30 amino acids, (e.g., furin cleavage site or glycine-serine linker, such as (GGGGS)ₙ). In some embodiments, the linker polypeptide consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the linker polypeptide consists of about 18 to 22 amino acids. In some embodiments, the linker polypeptide consists of 20 amino acids. In some embodiments, cleavable linkers include linkers that are cleaved by an enzyme exogenous to the modified cells in the population, for example, an enzyme encoded by a polynucleotide that is introduced into the cells by transfection or transduction, either at the same time or a different time as the polynucleotide that encodes the linker. In some embodiments, cleavable linkers include linkers that are cleaved by an enzyme endogenous to the modified cells in the population, including, for example, enzymes that are naturally expressed in the cell, and enzymes encoded by polynucleotides native to the cell, such as, for example, lysozyme.

2A Peptide Bond-Skipping Sequences

2A self-cleaving peptides, or 2A peptides, or “peptide bond-skipping” 2A sequences, are derived from, for example, many different viruses, including, for example, from foot-and-mouth disease virus, equine rhinitis A virus, Thosea asigna virus, etc. 2A peptides is a class of 18-22 aa-long peptides, which can induce the cleaving of the recombinant protein in the cell. When this type of sequence is placed within a cistron, between two polypeptides that are intended to be separated, the ribosome appears to skip a peptide bond. For example, in the case of Thosea asigna virus 2A sequence, the peptide bond between the Gly and Pro amino acid residues at the carboxy terminal “P-G-P” is omitted. This may, leave two to three polypeptides, for example, an inducible chimeric pro-apoptotic polypeptide and a chimeric antigen receptor, or, for example, a marker polypeptide and an inducible chimeric pro-apoptotic polypeptide. When this sequence is used, the polypeptide that precedes the 2A sequence may end up with additional amino acids at the carboxy terminus, including the Gly residue and any upstream residues in the 2A sequence. The peptide that is encoded 3′ of the 2A sequence may end up with additional amino acids at the amino terminus, including the Pro residue and any downstream residues following the 2A sequence. In some embodiments, the cleavable linker is a 2A polypeptide derived from porcine teschovirus-1 (P2A). In some embodiments, the 2A co-translational sequence is a 2A-like sequence. In some embodiments, the 2A co-translational sequence is T2A (Thosea asigna virus 2A), F2A (foot and mouth disease virus 2A), P2A (porcine teschovirus-1 2A), BmCPV 2A (cytoplasmic polyhedrosis virus 2A) BmIFV 2A (flacherie virus of B. mori 2A), or E2A (equine rhinitis A virus 2A). In some embodiments, the 2A co-translational sequence is T2A-GSG, F2A-GSG, P2A-GSG, or E2A-GSG. In some embodiments, the 2A co-translational sequence is selected from the group consisting of T2A, P2A and F2A. By “cleavable linker” is meant that the linker is cleaved by any means, including, for example, non-enzymatic means, such as peptide skipping, or enzymatic means. (Donnelly, M L 2001, J. Gen. Virol. 82:1013-25).

The 2A-like sequences are sometimes “leaky” in that some of the polypeptides are not separated during translation, and instead, remain as one long polypeptide following translation. One theory as to the cause of the leaky linker is that the short 2A sequence occasionally may not fold into the required structure that promotes ribosome skipping (a “2A fold”). In these instances, ribosomes may not miss the proline peptide bond, which then results in a fusion protein. To reduce the level of leakiness, and thus reduce the number of fusion proteins that form, a GSG (or similar) linker may be added to the amino terminal side of the 2A polypeptide; the GSG linker blocks secondary structures of newly translated polypeptides from spontaneously folding and disrupting the ‘2A fold.’

Therapeutic Applications

The immune cells enhanced with exogenous mitochondria provided herein (such as immune cells into which autologous mitochondria, allogeneic mitochondria, xenogeneic mitochondria, encapsulated mitochondria or mitochondria with genetic modification were transplanted) may be useful for the treatment of any disease or condition involving a target. If the application discloses a general application of immune cells (not binder-specific) then can use “tumor associated antigen” (“TAA”) as the target cell molecule. In some embodiments, the disease or condition is a disease or condition that can benefit from treatment with adoptive cell therapy. In some embodiments, the disease or condition is a tumor. In some embodiments, the disease or condition is a cell proliferative disorder. In some embodiments, the disease or condition is a cancer. In some embodiments, the disease or condition is a viral infection.

In some embodiments, provided herein is a method of treating a disease or condition in a subject in need thereof by administering to the subject an effective amount of an immune cell enhanced with exogenous mitochondria provided herein, e.g., immune cells previously transplanted with exogenous mitochondria ex vivo. In some embodiments, provided herein is a method of treating a disease or condition in a subject in need thereof by co-administering to the subject an effective amount of an immune cell together with exogenous mitochondria provided herein to the subject. In some aspects, the disease or condition is a cancer. In some aspects, the disease or condition is a viral infection.

Any suitable cancer may be treated with the immune cells enhanced with exogenous mitochondria provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin's lymphoma (HL), hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin's lymphoma (NHL), non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer (SCLC), small intestine cancer, soft tissue sarcoma, spinal cord tumor, stomach cancer, T cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

Combination Therapies

In some embodiments, the immune cells, such as T cells or CAR T cells, enhanced with exogenous mitochondria provided herein are administered with at least one additional therapeutic agent. Immune cells enhanced with exogenous mitochondria can include immune cells previously transplanted with exogenous mitochondria ex vivo, or immune cells co-administered with exogenous mitochondria such that exogenous mitochondria are transplanted into immune cells in vivo. Any suitable additional therapeutic agent may be administered with an immune cell enhanced with exogenous mitochondria provided herein. In some aspects, the additional therapeutic agent is selected from radiation, a cytotoxic agent, a chemotherapeutic agent, a cytostatic agent, an anti-hormonal agent, an EGFR inhibitor, an immunostimulatory agent, an anti-angiogenic agent, and combinations thereof.

In some embodiments, the additional therapeutic agent comprises an immunostimulatory agent.

In some embodiments, the immunostimulatory agent is an agent that blocks signaling of an inhibitory receptor of an immune cell, or a ligand thereof. In some aspects, the inhibitory receptor or ligand is selected from cytotoxic T-lymphocyte-associated protein 4 (CTLA-4, also known as CD152), programmed cell death protein 1 (also PD-1 or CD279), programmed death ligand 1 (also PD-L1 or CD274), transforming growth factor beta (TGFβ), lymphocyte-activation gene 3 (LAG-3, also CD223), Tim-3 (hepatitis A virus cellular receptor 2 or HAVCR2 or CD366), neuritin, B- and T-lymphocyte attenuator (also BTLA or CD272), killer cell immunoglobulin-like receptors (KIRs), and combinations thereof. In some aspects, the agent is selected from an anti-PD-1 antibody (e.g., pembrolizumab or nivolumab), and anti-PD-L1 antibody (e.g., atezolizumab), an anti-CTLA-4 antibody (e.g., ipilimumab), an anti-TIM3 antibody, carcinoembryonic antigen-related cell adhesion molecule 1 (CECAM-1, also CD66a) and 5 (CEACAM-5, also CD66e), vset immunoregulatory receptor (also VISR or VISTA), leukocyte-associated immunoglobulin-like receptor 1 (also LAIR1 or CD305), CD160, natural killer cell receptor 2B4 (also CD244 or SLAMF4), and combinations thereof. In some aspects, the agent is pembrolizumab. In some aspects, the agent is nivolumab. In some aspects, the agent is atezolizumab.

In some embodiments, the additional therapeutic agent is an agent that inhibits the interaction between PD-1 and PD-L1. In some aspects, the additional therapeutic agent that inhibits the interaction between PD-1 and PD-L1 is selected from an antibody, a peptidomimetic and a small molecule. In some aspects, the additional therapeutic agent that inhibits the interaction between PD-1 and PD-L1 is selected from pembrolizumab (Keytruda™), nivolumab (Opdivo™) atezolizumab (Tecentriq™), avelumab (Bavencio™), pidilizumab, durvalumab, BMS-936559, sulfamonomethoxine 1, and sulfamethizole 2. In some embodiments, the additional therapeutic agent that inhibits the interaction between PD-1 and PD-L1 is any therapeutic known in the art to have such activity, for example as described in Weinmann et al. (Weinmann, 2016, “Corrigendum: Cancer Immunotherapy: Selected Targets and Small-Molecule Modulators”, ChemMedChem 11:1576), incorporated by reference in its entirety. In some embodiments, the agent that inhibits the interaction between PD-1 and PD-L1 is formulated in the same pharmaceutical composition an antibody provided herein. In some embodiments, the agent that inhibits the interaction between PD-1 and PD-L1 is formulated in a different pharmaceutical composition from an antibody provided herein. In some embodiments, the agent that inhibits the interaction between PD-1 and PD-L1 is administered prior to administration of an antibody provided herein. In some embodiments, the agent that inhibits the interaction between PD-1 and PD-L1 is administered after administration of an antibody provided herein. In some embodiments, the agent that inhibits the interaction between PD-1 and PD-L1 is administered contemporaneously with an antibody provided herein, but the agent and antibody are administered in separate pharmaceutical compositions.

In some embodiments, the immunostimulatory agent is an agonist of a co-stimulatory receptor of an immune cell. In some aspects, the co-stimulatory receptor is selected from GITR, OX40, ICOS, LAG-2, CD27, CD28, 4-1BB, CD40, STING, a toll-like receptor, RIG-1, and a NOD-like receptor. In some embodiments, the agonist is an antibody.

In some embodiments, the immunostimulatory agent modulates the activity of arginase, indoleamine-2 3-dioxygenase, or the adenosine A2A receptor.

In some embodiments, the immunostimulatory agent is a cytokine. In some aspects, the cytokine is selected from IL-2, IL-5, IL-7, IL-12, IL-15, IL-21, and combinations thereof. In some aspects, the cytokine is IL-2.

In some embodiments, the immunostimulatory agent is an oncolytic virus. In some aspects, the oncolytic virus is selected from a herpes simplex virus, a vesicular stomatitis virus, an adenovirus, a Newcastle disease virus (NDV), a vaccinia virus, and a maraba virus.

Further examples of additional therapeutic agents include a taxane (e.g., paclitaxel or docetaxel); a platinum agent (e.g., carboplatin, oxaliplatin, and/or cisplatin); a topoisomerase inhibitor (e.g., irinotecan, topotecan, etoposide, and/or mitoxantrone); folinic acid (e.g., leucovorin); or a nucleoside metabolic inhibitor (e.g., fluorouracil, capecitabine, and/or gemcitabine). In some embodiments, the additional therapeutic agent is folinic acid, 5-fluorouracil, and/or oxaliplatin. In some embodiments, the additional therapeutic agent is 5-fluorouracil and irinotecan. In some embodiments, the additional therapeutic agent is a taxane and a platinum agent. In some embodiments, the additional therapeutic agent is paclitaxel and carboplatin. In some embodiments, the additional therapeutic agent is pemetrexed. In some embodiments, the additional therapeutic agent is a targeted therapeutic such as an EGFR, RAF or MEK-targeted agent.

The additional therapeutic agent may be administered by any suitable means. In some embodiments, a medicament provided herein, and the additional therapeutic agent are included in the same pharmaceutical composition. In some embodiments, an antibody provided herein, and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some aspects, administration of an antibody provided herein, and the additional therapeutic agent occur within about one month of each other. In some aspects, administration of an antibody provided herein, and the additional therapeutic agent occur within about one week of each other. In some aspects, administration of an antibody provided herein, and the additional therapeutic agent occur within about one day of each other. In some aspects, administration of an antibody provided herein, and the additional therapeutic agent occur within about twelve hours of each other. In some aspects, administration of an antibody provided herein, and the additional therapeutic agent occur within about one hour of each other.

Methods of Use

The present specification provides methods to deliver isolated mitochondria or pharmaceutical compositions of isolated mitochondria ex vivo to the cells of a patient or allogeneic donor and/or in vivo to tissues of a patient. Without wishing to be bound by theory, mitochondria are taken up by tissue cells or cultured cells through an actin-dependent endocytosis, thereby providing a way to deliver the pharmaceutic composition directly into the cells. In a non-limiting illustrative example, mitochondria are transplanted into the target immune cells by e.g., co-incubation of mitochondria (10⁷/well) with the cells (50,000/well) in culture medium over the period of 2-24 hours. One skilled in the art can recognize the dosage of mitochondria administered to immune cells ex vivo or to tissues of a patient in vivo may be varied based on the intended outcome in terms of enhancing the target immune cell or cells, such as optimization of viability, efficacy, activity, survival, endurance and/or toxicity. In the ex vivo delivery of mitochondria to immune cells, e.g., through co-incubation, the dosage of mitochondria may be between 0.2 mitochondria per target-cell and 5,000 mitochondria per target cell. In delivery of mitochondria in vivo, to the tissue of a patient, between 1 mitochondrion and 10⁷ Mitochondria per 1 mL may be delivered.

The present disclosure contemplates a composition comprising enhanced immune cells (αβT cells, γδT cells, Treg cells, CAR-T cells, NK cells, CAR-NK cells, NK T cells, macrophages, CAR-macrophages, neutrophils, CAR-neutrophils, etc.), wherein the cells comprise or are enhanced by exogenous mitochondria, which may be autologous mitochondria, allogeneic mitochondria, xenogeneic mitochondria, encapsulated mitochondria or autogenous mitochondria with genetic modification. These cells can be either any effector cells known in the art with anti-tumor activity or immunosuppressive immune cells able to prevent autoimmunity. Accordingly, the present specification provides methods to deliver immune cells comprising or enhanced by exogenous mitochondria, or pharmaceutical compositions of immune cells comprising or enhanced by exogenous mitochondria, to the cells and/or tissues of a patient or cells derived from an allogeneic donor. The immune cells comprising or enhanced by exogenous mitochondria can be used to treat a variety of diseases, including but not limited to various forms of cancer, tumors and autoimmune disease.

In some embodiments, preparation of CAR T cells can include the following steps:
1. Collecting T lymphocytes from patient's blood by leukapheresis.
2. Enrichment of T cells by density gradient centrifugation, elutriation, and immunomagnetic bead selection.
3. Gene modification using electroporation, retroviral/lentiviral transduction, or nuclease-meditated genome editing (e.g., introduction of CAR gene into the genome of the target cell).
4. Activation and expansion of CAR-T cells via polyclonal activation through artificial antigen presenting systems (anti-CD8/anti-CD28 immunomagnetic beads/LV-APCs) using methods known in the art.
   Consistency is generally achieved through standardization and validation of raw materials and protocols according to cGMPs (current good manufacturing practices).
5. Quality Assurance—testing for viability, phenotyping, gram staining, endotoxin, and bacterial, fungal, and mycoplasma contaminants pursuant to the FDA guidelines using methods known in the art.
6. Formulation and Administration—testing for clinically prescribed dosage and route of administration using methods known in the art.
   Therapeutic cell preservation, packaging, transport, receipt, and administration generally should maintain product stability and chain of custody.

In a particular embodiment, mitochondria preparations are delivered to immune cells (1) before, (2) concurrently with, or (3) after genetic modification (e.g., introduction of the CAR gene) is performed. In a particular embodiment, mitochondria preparations are delivered ex vivo to immune cells (1) before, (2) concurrently with, or (3) after ex vivo genetic modification (e.g., introduction of the CAR gene) is performed, such as in methods including ex vivo genetic modification. In a particular embodiment, mitochondria preparations are delivered ex vivo to immune cells before in vivo genetic modification (e.g., introduction of the CAR gene) is performed (e.g., in vivo virally mediated genetic modification). Without wishing to be bound by theory, Step (1) is typically important for regeneration of the autologous T cells (exhausted or senescent T cells) taken from the immunocompromised cancer patients. The mitochondria can be co-incubated with the cells ex vivo at ratios between 0.2:1 to 5000:1, for example at ratios of 0.2:1, 0.5:1, 1:1, 10:1, 50:1, 100:1, 200:1, 500:1, 1000:1 or 5000:1.

In order to boost immune cell activity, such as CAR-T cell activity, in vivo, mitochondria can also be delivered (4) along with the immune cells into a patient. In a particular embodiment, mitochondria preparations are delivered in vivo to immune cells (1) before, (2) concurrently with, or (3) after in vivo genetic modification (e.g., introduction of the CAR gene) is performed (e.g., in vivo virally mediated genetic modification). In a particular embodiment, mitochondria preparations are delivered in vivo to immune cells after ex vivo genetic modification (e.g., introduction of the CAR gene) is performed. In a particular embodiment of the present invention, the CAR-T cells or other immune cells are delivered via a systemic (intravenous) infusion while mitochondria are delivered (5) via intratumoral injection, (6) intraorgan injection, (7) intra-tissue injection, or (8) through the organ-specific or tissue-specific vasculature.

### EXAMPLES

The following are examples of methods and compositions of the invention. It is understood that various other embodiments may be practiced, given the general description provided herein.

### Example 1a: Isolating Mitochondria from Tissue Samples or Cultured Cells

Experiments were performed to isolate mitochondria from tissue samples or cultured cells.

Preparation

The following solutions were prepared to isolate intact, viable, respiration-competent mitochondria. To successfully isolate mitochondria using the present methods, solutions and tissue samples should be kept on ice to preserve mitochondrial viability. Even when maintained on ice, isolated mitochondria will exhibit a decrease in functional activity over time (Olson et al., J Biol Chem 242:325-332, 1967). The following solutions should be prepared in advance if possible:
1 M K-HEPES Stock Solution (adjust pH to 7.2 with KOH).
0.5 M K-EGTA Stock Solution (adjust pH to 8.0 with KOH).
1 M KH₂PO₄ Stock Solution.
1 M MgCl₂ Stock Solution.
Homogenizing Buffer (pH 7.2): 300 mM sucrose, 10 mM K-HEPES, and 1 mM K-EGTA. Stored at 4° C.
Respiration Buffer: 250 mM sucrose, 2 mM KH₂PO₄, 10 mM MgCl₂, 20 mM K-HEPES Buffer (pH 7.2), and 0.5 mM K-EGTA (pH 8.0). Stored at 4° C.
10×PBS Stock Solution: 80 g of NaCl, 2 g of KCl, 14.4 g of Na₂HPO₄, and 2.4 g of KH₂PO₄ were dissolved in 1 L double distilled H₂O (pH 7.4).
1×PBS was prepared by pipetting 100 mL 10×PBS into 1 L double distilled H₂O.Subtilisin A Stock was prepared by weighing out 4 mg of Subtilisin A into a 1.5 mL microfuge tube. Stored at −20° C. until use.

Isolation of Mitochondria from Tissue

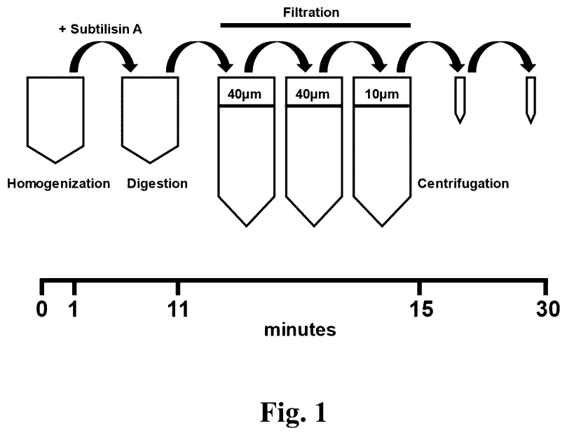

A scheme outlining the procedural steps in the isolation of mitochondria using tissue dissociation and differential filtration is shown in FIG. 1. Two, 6 mm biopsy fresh sample punches taken from the skeletal muscles were transferred to 5 mL of Homogenizing Buffer in a gentleMACS C Tube (Miltenyi Biotec, Somerville, MA) and the samples were homogenized using the gentleMACS™ Dissociator's (Miltenyi Biotec) 1-minute homogenization program. Subtilisin A stock solution (250 μL) was added to the homogenate in the gentleMACS C tube and incubated on ice for 10 minutes. The homogenate was centrifuged at 750×g for 4 minutes (as an optional step). Afterwards, the homogenate was filtered through a pre-wetted 40 μm mesh filter in a 50 mL conical centrifuge tube on ice. The filtrate was re-filtered through a new pre-wetted 40 μm mesh filter in a 50 mL conical centrifuge on ice. The filtrate was re-filtered again through a new pre-wetted 10 μm mesh filter in a 50 mL conical centrifuge tube on ice. The filtrate was re-filtered through a new pre-wetted 6 μm mesh filter in a 50 mL conical centrifuge tube on ice. The resulting filtrate was either used immediately or concentrated by centrifugation. In the case of concentration, the filtrate was transferred to 1.5 mL microfuge tubes and centrifuged at 9000×g for 10 minutes at 4° C. The supernatant was removed, and the pellets containing mitochondria were re-suspended, and combined in 1 mL of Respiration Buffer.

Isolation of Mitochondria from Cultured Cells

Mitochondria were also isolated from the cultured cells, for example, from human cardiac fibroblast (HCF) cell line (obtained from ScienCell Research Laboratories, Carlsbad, CA).

Culture of the Human Cardiac Fibroblast (HCF) Cells

Human cardiac fibroblasts (HCF) were maintained in Fibroblast Medium-2 containing fetal bovine serum, fibroblast growth supplement-2, and antibiotic (penicillin/streptomycin) solution according to the supplier's directions (ScienCell). The cells were maintained as a monolayer at 37° C. in humidified atmosphere of 5% CO₂ and were passaged when 80% confluence was reached.

Preparation of the Human Cardiac Fibroblast (HCF) Cells

HCF cells from two flasks (T150) at a confluency of 80% were washed once with PBS. Then trypsin was used to detach the cells according to the supplier instructions (ScienCell Research Laboratories, Carlsbad, CA). The reaction was stopped by adding trypsin neutralizing solution according to the supplier's instructions (ScienCell Research Laboratories, Carlsbad, CA). The cells were collected in a 50 ml centrifuge tube and centrifuged for 5 minutes at 1000 rpm (190×g). The supernatant was discarded and three washes with 1×PBS were performed in total.

Preparation of culture cells different from HCF, should be done according to the manufacturer's instructions. Of note, the cells used as the source of mitochondria can be adherent, semi-adherent or in suspension.

The mitochondria isolation procedure was essentially the same as the procedure for isolating mitochondria from the tissue samples, except that human fibroblasts were used rather than biopsy samples.

Alternatively, mitochondria could be isolated by repetitive centrifugation (Kesner et al., 2016, “Characteristics of Mitochondrial Transformation into Human Cells”, Sci Rep 6:26057). In brief, the cells were collected by trypsinization, suspended in PBS, and centrifuged (5 minutes, 250×g) twice. Mitochondrial isolation procedures were performed at 4° C. or on ice. The centrifuged cells were re-suspended in mitochondrial isolation buffer (320 mM sucrose, 5 mM Tris-HCl, pH 7.4, 2 mM EGTA), and homogenized with a Dounce homogenizer. Nuclei and cell debris were removed by two centrifugations at 3000×g for 5 minutes and the supernatant was collected (optional step). The supernatant was then centrifuged at 12,000×g for 10 minutes, and the mitochondrial pellet was re-suspended in mitochondrial isolation buffer. Mitochondrial concentration was determined by Bradford assay.

Mitochondrial Number

Viable mitochondrial number was determined by labeling an aliquot (10 μL) of isolated mitochondria with MitoTracker Orange CMTMRos (5 μmol/L; Thermo Fisher Scientific). Aliquots of labeled mitochondria were spotted onto slides and counted using a spinning disk confocal microscope with a 63×C-apochromat objective (1.2 W Korr/0.17 NA, Zeiss). Mitochondria were counterstained with the mitochondria-specific dye MitoFluor Green (Thermo Fisher Scientific). Appropriate wavelengths were chosen for measurement of autofluorescence and background fluorescence with use of unstained cells and tissue. Briefly, 1 μL of labeled mitochondria was placed on a microscope slide and covered. Mitochondrial number was determined at low (×10) magnification covering the full specimen area using MetaMorph Imaging Analysis software.

### Example 1b: Isolating Mitochondria from Cultured Cells

Experiments were performed to isolate mitochondria from cultured cells.

Preparation

The following solutions were prepared to isolate intact, viable, respiration-competent mitochondria. To successfully isolate mitochondria using the present methods, solutions and tissue samples should be kept on ice to preserve mitochondrial viability. Even when maintained on ice, isolated mitochondria will exhibit a decrease in functional activity over time (Olson et al., J Biol Chem 242:325-332, 1967). The following solutions should be prepared in advance if possible:
1 M K-HEPES Stock Solution (adjust pH to 7.2 with KOH).
0.5 M K-EGTA Stock Solution (adjust pH to 8.0 with KOH).
Homogenizing Buffer (pH 7.2): 300 mM sucrose, 10 mM K-HEPES, and 1 mM K-EGTA. Stored at 4° C.
1×PBS (ThermoFisher, 10010031)
Subtilisin A Stock was prepared by weighing out 2 mg of Subtilisin A into a 1.5 mL microfuge tube. Stored at −20° C. until use. Prepared at 2 mg/ml in Homogenizing Buffer.

Culture of the Human Cardiac Fibroblast (HCF) Cells

Human cardiac fibroblasts (HCF) (obtained from ScienCell Research Laboratories, Carlsbad, CA) were cultured as described in Example 1a, with the only difference that the cells were passaged when 90% confluence was reached.

Preparation of culture cells different from HCF, should be done according to the manufacturer's instructions. Of note, the cells used as the source of mitochondria can be adherent, semi-adherent or in suspension.

Isolation of Mitochondria from Cultured Cells

Mitochondria were also isolated from cultured cells, for example, from human cardiac fibroblast (HCF) cell line. The preparation of HCF cells was done according to the of Example 1a. The HCF cells from each flask were then transferred to 5 mL of Homogenizing Buffer in a gentleMACS C Tube (Miltenyi Biotec, Somerville, MA) and the samples were homogenized using the gentleMACS™ Dissociator's (Miltenyi Biotec) 1-minute homogenization program. Subtilisin A stock solution (250 μL) was added to the homogenate in the gentleMACS C tube and incubated on ice for 10 minutes. The homogenate was filtered through a pre-wetted 40 μm mesh filter in a 50 mL conical centrifuge tube on ice. The filtrate was re-filtered through a new pre-wetted 40 μm mesh filter in a 50 mL conical centrifuge on ice. The filtrate was re-filtered again through a new pre-wetted 10 μm mesh filter in a 50 mL conical centrifuge tube on ice. Optionally, the filtrate was re-filtered again through a new pre-wetter 5 μm mesh filter in a 50 mL conical centrifuge tube on ice. The resulting filtrate was either used immediately or concentrated by centrifugation. In the case of concentration, the filtrate was transferred to 1.5 mL microfuge tubes and centrifuged at 9500×g for 5 minutes at 4° C. Three washes were performed at the same centrifugation speed.

Quantification of Isolated Mitochondria

The isolated mitochondria were suspended in the Homogenizing Buffer of Example 1b and kept on ice until use. Mitochondria quantity, in preparation for varying dosage administration, was measured using a Qubit™ Fluorometer (ThermoFisher Scientific/Invitrogen), employing the Qubit™ Protein Assay kit in accordance with the manufacturer's instructions. For the protein concentration measurement, the mitochondria were resuspended in PBS (ThermoFisher, 10010031). The mitochondria dosage was estimated in terms of protein content expressed in g.

### Example 2: T Cell Isolation, Activation and Culture

CD8⁺ T cells were isolated from buffy coats of healthy donors. Peripheral blood mononuclear cells (PBMC) were collected by density gradient centrifugation using Ficoll Paque plus (Cytiva, 17144002) according to the manufacturer's instructions. Human CD8⁺ T cells were harvested from the PBMCs using the EasySep™ Human CD8⁺ T Cell Isolation Kit (Stemcell, 17953) and The Big Easy” EasySep™ Magnet (Stemcell, 18001). Isolated CD8⁺ T cells were activated with Dynabeads Human T-Activator CD3/CD28 (ThermoFisher, 111.32D), in a 1 to 1 ratio, in presence of 100 U/ml of recombinant human IL-2 (Peprotech, 200-02). CD8⁺ T cells were cultured in RPMI 1640 medium GlutaMAX™ Supplement 500 ml (ThermoFisher, 61870010), supplemented with 1% L-glutamine (ThermomFisher, 25030024), 1% penicillin-streptomycin (10,000 U/mL, Gibco, 15140122), 1% non-essential amino acid (NEAA, ThermoFisher, 11140050), 1% sodium pyruvate (ThermoFisher, 11360070), 10% fetal bovine serum and 0.1% 2β-mercaptoethanol (Gibco, 31350-010). CD8⁺ T cells were plated at 0.5 Million of cells/mL and split when the cells reached a confluency of 2 Million cells/mL or when the medium was turning yellow.

### Example 3: T Cell Transplantation

CD8⁺ T cells were plated at 0.5 Million cells/mL in a 24 well plate 24 h prior to mitochondria transplantation. When the mitochondria were isolated, CD8⁺ T cells were collected and centrifuged for 5 minutes at 1500 rpm (430×g). The supernatant was discarded and the cells were resuspended in fresh T cell medium at the concentration of 1 Million cell/100 μL. The T cell medium is described under Example 2.

Transplanted CD8⁺ T cells were incubated for 4 h with isolated mitochondria in a range of 10 μg to 100 μg of protein per 1 Million of CD8⁺ T cells in a final volume of 200 μL of T cell medium in each well of the 24 well plate. 4 h post co-incubation of exogenous mitochondria and CD8⁺ T cells, 1.8 ml of fresh T cell medium was added per well.

### Example 4a: Mitochondria Labeling and Internalization

The isolated mitochondria are labeled with pHrodo red particle label (Life Technologies, Grand Island, NY) for 10 minutes at 4° C. and then washed four times in respiration buffer (250 mM sucrose, 2 mM KH₂PO₄, 10 mM MgCl₂, 20 mM K⁺-HEPES buffer, pH 7.2, 0.5 mM K⁺-EGTA, pH 8.0, 5 mM glutamate, 5 mM malate, 8 mM succinate, 1 mM ADP). The labeled mitochondria are resuspended in fresh respiration buffer and the last wash supernatant is saved. The labeled mitochondria are co-incubated with isolated T cells. At the conclusion of each time point, the media is removed, and the cells are washed four times with 1×PBS and 200 μL of fresh medium was added to each well. Control cells were co-incubated with the last pHrodo wash. Mitochondrial internalization is determined using ImageJ 1.48 software.

ATP Assay

ATP content is determined using the ATPlite Luminescence ATP Detection Assay System (Perkin Elmer, Waltham, MA). All assays are performed in the absence of fluorescent dyes, since these dyes may interfere with mitochondrial function.

The results demonstrate that T cells co-incubated with mitochondria are internalized in a time-dependent manner. The internalized mitochondria significantly increase the T-cell ATP content as compared to control.

### Example 4b: Mitochondria Labeling and Internalization

### Example 4b.1—T Cell Transplantation with Stained Isolated Mitochondria

CD8⁺ T cells were plated at 0.5 Million cells/mL in a 24 well plate 24 h prior to mitochondria transplantation. Mitochondria were isolated according to procedure described under Example 1b. Mitochondria were then stained for 10 to 15 minutes at 37° C. with Mitotracker Red CMXRos (ThermoFisher, M7512) and Mitotracker Green FM (ThermoFisher, M7514) at 200 nM in the Homogenizing Buffer of Examples 1b. Three washes of the stained mitochondria were performed with Homogenizing Buffer of Examples 1b at 9500×g for 5 minutes at 4° C. and the supernatant of the last wash was saved as a control. CD8⁺ T cells were collected and centrifuged for 5 minutes at 1500 rpm (430×g). The supernatant was removed and the cells were resuspended in fresh T cell medium at 1 Million cells/100 μL. The T cell medium is described under Example 2. Stained mitochondria were (immediately) added to the T cells to obtain a final volume of 200 μL per well of a 24 well plate. The last wash of the stained mitochondria was added in an equivalent volume to the control non-transplanted CD8⁺ T cells. The integration of the stained mitochondria was evaluated by flow cytometry (e.g., data acquired with FACSLyric (BD Biosciences)) or by fluorescence microscopy (Keyence microscope, BZ-X810) from 5 minutes to 24 h post transplantation. In case of a co-incubation of exogenous mitochondria and CD8⁺ T cells longer than 4 h, 1.8 ml of fresh T cell medium was added per well.

### Example 4b.2 Staining Post Mitochondria Transplantation

Transplanted CD8⁺ T cells were incubated for 4 h with isolated mitochondria in a range of 10 μg to 100 μg of protein per 1 Million of CD8⁺ T cells in a final volume of 200 μL of T cell medium in each well of the 24 well plate. 4 h post co-incubation of exogenous mitochondria and CD8⁺ T cells, 1.8 ml of fresh T cell medium was added per well. Mitochondrial respiration and mass were evaluated in transplanted cells 24 h post co-incubation. The dyes Mitotracker Red CMXRos (ThermoFisher, M7512) and Mitotracker Green FM (ThermoFisher, M7514) were diluted to a final concentration of 100 nM in RPMI 1640 medium, no phenol red (ThermoFisher, 11835030), supplemented with 1% penicillin-streptomycin (10,000 U/mL, Gibco, 15140122), 5% fetal bovine serum. 100 μl of the staining was added per 1 Million of CD8⁺ T cells and the staining was performed for 15 minutes at 37° C. The cells were then washed twice with FACS buffer (1×PBS (ThermoFisher, 10010031), 2% FBS, 1% EDTA 0.5M (Sigma-Aldrich, E6758)) at 1500 rpm (430×g) for 5 minutes. The supernatant was discarded and the CD8⁺ T cells were resuspended in 300 μL of FACS buffer and acquired on a FACS machine (FACSLyric, BD Biosciences).

### Example 5: Transplantation of Exogenous Mitochondria into T Cells

Two experimental set-ups were employed to determine the uptake of exogenous mitochondria and its dosage-dependency when transplanted into CD8⁺ T cells isolated from healthy donors and subsequently cultivated. First, experiments employing flow cytometry and second, experiments employing fluorescent microscopy.

Procedure
(i) T cell isolation, activation, and culture was performed as described in Example 2.
(ii) Mitochondria isolation: Mitochondria were isolated from human cardiac fibroblasts (HCF) as previously described in Example 1b. The isolated mitochondria were suspended in the Homogenizing Buffer of Example 1b and kept on ice until use. Mitochondria quantity, in preparation for varying dosage administration, was measured using a Qubit™ Fluorometer (ThermoFisher Scientific/Invitrogen), employing the Qubit™ Protein Assay Kit in accordance with the manufacturer's instructions. The mitochondria dosage is estimated in terms of protein content expressed in g.
(iii) T cell transplantation with stained isolated mitochondria was performed as described in Example 4b.1. The integration of the stained mitochondria was evaluated by flow cytometry (e.g., data acquired with FACSLyric (BD Biosciences)), and by fluorescence microscopy (Keyence microscope, BZ-X810) from 5 min to 24 h post transplantation.

Results

(i) Dose-Dependent Integration of Mitochondria Confirmed by Flow Cytometry and Fluorescent Microscopy

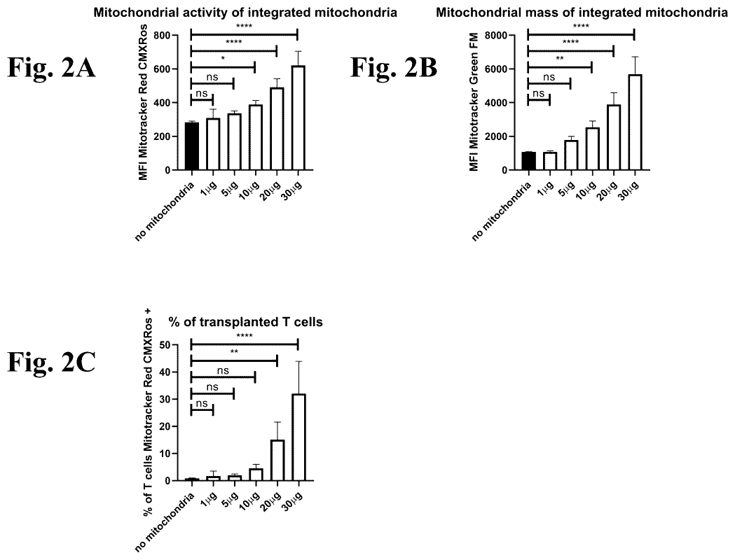

To investigate the dose-dependent uptake of mitochondria in CD8⁺ T cells, stained exogenous mitochondria were transplanted into CD8⁺ T cells at dosage levels varying between 1 μg-30 μg of mitochondria per 1 million CD8⁺ T cells. CD8⁺ T cells were extracted from the blood of five different donors as described above and subsequently cultivated separately for each donor. The exogenous mitochondria were isolated from human cardiac fibroblasts and stained with Mitotracker Red CMXRos and Mitotracker Green FM, as described in Example 4b.1. Subsequently, the CD8⁺ T cells from each donor were treated with the stained exogenous mitochondria at the following dosage levels: 1 g, 5 g, 10 g, 20 g, and 30 g per 1 million CD8⁺ T cells in a final volume of 200 μl. Four hours post incubation, the expression of Mitotracker Red CMXRos and Mitotracker Green FM was assessed by flow cytometry, using a FACSLyric (BD Biosciences) and by fluorescent microscopy, using a Keyence microscope (BZ-X810) as per the manufacturer's instructions. As shown in FIGS. 2A-2D, there is a clear dose dependent expression of Mitotracker Red CMXRos and Mitotracker Green FM in the treated CD8⁺ T cells. The transplantation efficiency of CD8⁺ T cells is increased in a dose-dependent manner. Both, mitochondrial activity and mitochondrial mass of the integrated mitochondria are significantly increased in the treated cells when compared to the CD8⁺ T cells that did not received exogenous mitochondria. A significant increase of both mitochondrial activity and mitochondrial mass was shown when starting from 10 g of mitochondria.

### Example 6: Transplanting Exogenous Mitochondria into T Cells to Improve Proliferation

An experiment was performed to assess the impact of transplanting exogenous mitochondria on CD8⁺ T cell proliferation capacity. Specifically, T cell expansion was evaluated 24 h, 48 h, 72 h and 140 h post mitochondria transplantation. The cells were diluted in Trypan Blue solution 0.4% (ThermoFisher, 15250061) and living cells (white) were counted, whereas dead cells (blue) were excluded from the analysis. The fold change was calculated for each donor at each time point between the number of cells in the control and treated groups, which received 30 μg of mitochondria. Overall proliferation was shown to be enhanced in CD8⁺ T cells transplanted with exogenous mitochondria.

Procedure
(i) T cell isolation, activation and culture as described in Example 2.
(ii) Mitochondria isolation as described in Example 1b. The mitochondria isolation was performed in a sterile environment and the overall expansion was determined.
(iii) Mitochondria Transplantation: CD8⁺ T cells were plated at 0.5 Million cells/mL in a 24 well plate 24 h prior to mitochondria transplantation. Mitochondria were isolated from human cardiac fibroblasts, as previously described in Example 1b. When the mitochondria were isolated, CD8⁺ T cells were collected and centrifuged for 5 minutes at 1500 rpm (430×g). The supernatant was discarded and the cells were resuspended in fresh T cell medium at the concentration of 1 Million cell/100 μL. Mitochondria were added to the T cells to obtain a final volume of 200 μL per well of a 24 well plate. 4 h post incubation, 1.8 mL of fresh T cell medium was added per well. The T cell medium is described in Example 2. The amount of mitochondria for the treatment of the T cell was 30 μg.

Results: Enhanced Proliferation of Transplanted CD8⁺ T Cells

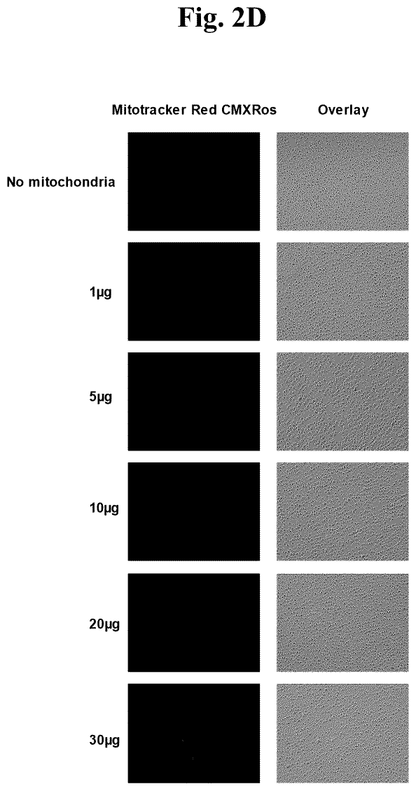

As depicted in FIG. 3, the treatment of CD8⁺ T cells with exogenous mitochondria derived from human cardiac fibroblasts significantly improved proliferation.

### Example 7: Assessing the Impact of Mitochondria Transplantation on CD8⁺ T Cell Activity-Potential

To assess the impact of mitochondria transplantation on the target cells' energy metabolism and activity-potential, a Mito Stress Test (Agilent Seahorse, P/N 103015-100) was performed to measure basal oxygen consumption rate (OCR) and maximal OCR upon exposure to FCCP. FCCP (Trifluoromethoxy-carbonylcyanide-phenylhydrazone; Carbonyl-cyanide-4-(trifluoromethoxy)-phenylhydrazone) is a potent uncoupler of oxidative phosphorylation in mitochondria that inhibits ATP synthesis by disrupting the electrochemical gradient. A Seahorse XFe96 Analyzer (Vigilant) was used.

Procedure
(i) T cell isolation, activation and culture was performed as described in Example 2.
(ii) Mitochondria isolation: Mitochondria were isolated from Human Cardiac Fibroblast (HCF) according to the procedure described in Example 1b.
(iii) Quantification of isolated mitochondria: The mitochondria dosage was estimated in terms of protein content expressed in g, according to the procedure described in Example 1b.
(iv) T cell transplantation according to the procedure of Example 3. An amount of 30 μg of mitochondria was transplanted in the CD8⁺ T cells.
(v) Seahorse assay for assessment of cell-metabolism: Transplanted CD8⁺ T cells were analyzed in a Seahorse XFe96 Analyzer according to the supplier's instructions (Agilent). T cells were plated at 0.5 Million cells/mL 24 h prior to transplantation with 30 μg of isolated mitochondria per million of T cells. 4 h post transplantation, fresh T cell medium supplemented with 100 U/mL of recombinant IL-2 was added per well of the 24 well plate. The cartridges (Agilent Seahorse, P/N 102416-100) were hydrated with 250 μL of ultrapure water and incubated overnight at 37° C. in an oven (VWR, 390-0384). The day of the assay, the ultrapure water used to hydrate the cartridges was replaced with 250 μL of calibrant (Agilent Seahorse, P/N 102416-100). The wells of the cell culture microplate (Agilent Seahorse, P/N 102416-100) were coated overnight at 4° C. with 50 μL of Cell-Tak (Corning, 354241) diluted 1/50 in PBS. 24 h post transplantation, CD8⁺ T cells were centrifuged at 200×g for 5 minutes, the supernatant was discarded, and the cells were counted and resuspended in Seahorse XF RPMI pH7.4 (Agilent Seahorse, P/N 103576-100) supplemented with 10 mM glucose (Agilent Seahorse, 103577-100), 2 mM glutamine (Agilent Seahorse, 103579-100) and 1 mM pyruvate (Agilent Seahorse, 103578-100). The cell culture plate was washed once with PBS and CD8⁺ T cells were plated at 300′000 cells/50 μL. The plate was centrifuged at 100×g for 5 minutes without brakes, 130 μL of warm Seahorse XF RPMI pH7.4 medium supplemented was added per well and the plate was then incubated for 1 h at 37° C. in an oven. The drugs from the Seahorse XF Mito Stress kit (Agilent Seahorse, P/N 103015-100) were prepared according to the supplier's instruction. The final concentration of 2 μM of Oligomycin, 1.5p M of FCCP and 0.5p M of Rotenone/Antimycin A were used to fill the cartridges, which were then loaded to the Seahorse XFe96 machine to enable calibration. The cell culture plate containing the transplanted T cells was loaded in the Seahorse XFe96 machine and the XP cell Mito Stress Test program was selected.

Results: Treated CD8⁺ T Cells Exhibit Increased Basal and Maximal Oxygen Consumption Rate

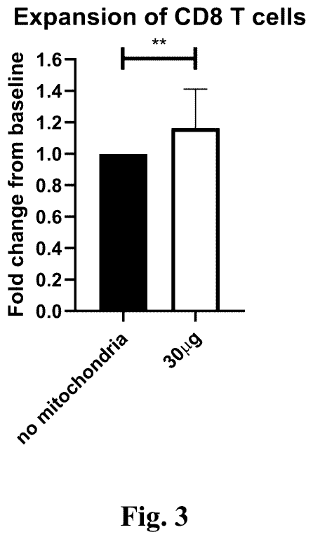

The Seahorse XFe96 in the XP cell Mito Stress Test program quantifies over time oxygen consumption rate of CD8⁺ T cells at basal level and upon treatment with oligomycin and subsequently with FCCP. The basal oxygen consumption rate measures the mitochondrial respiration (Gerritje J. W. et al., Current Protocols in Immunology 3.16B-1-3.16B 14, April 2016), while the reaction of the cells to FCCP allows conclusions to be drawn on the cells' level of maximum oxygen consumption capacity activity. As illustrated in FIG. 4A and FIG. 4C, the basal oxygen consumption rate is significantly enhanced in CD8⁺ T cells that were treated with exogenous mitochondria. Likewise, the cells' oxygen consumption rate upon exposure to FCCP is significantly increased upon mitochondria transplantation into the CD8⁺ T cells—illustrated in FIG. 4B and FIG. 4D.

### Example 8: Transplanting Exogenous Mitochondria into CD8⁺ T Cells to Improve Energy Metabolism

An experiment was performed to assess the functional impact of transplanting exogenous mitochondria into CD8⁺ T cells. Mitochondrial fitness in treated cells was assessed 24 hours post transplantation. Mitochondrial activity and mass were measured as mean fluorescence intensity via flow cytometry (e.g., FACSLyric (BD Biosciences)) by Mitotracker Red CMXRos (ThermoFisher, M7512) and Mitotracker Green FM (ThermoFisher, M7514) respectively. The ratio of Mitotracker Red CMXRos over Mitotracker Green FM calculates the mitochondrial activity per mitochondrial mass within the CD8⁺ T cells and has been established as an indication of mitochondrial fitness (Pendergrass et al., Cytometry Part A 61A:162-169 (2004)). Mean fluorescence intensity was measured via flow cytometry (e.g., FACSLyric (BD Biosciences)). It was shown that mitochondrial fitness of CD8⁺ T cells was improved by treating the cells with exogenous mitochondria.

Procedure
(i) T cell isolation, activation and culture as described in Example 2.
(ii) Mitochondria isolation as described in Example 1b.
(iii) Mitochondria transplantation and Staining post mitochondria transplantation: it was done according to the procedure described in Example 4b.2.

Results: Improvement of Mitochondria Fitness in CD8⁺ T Cells

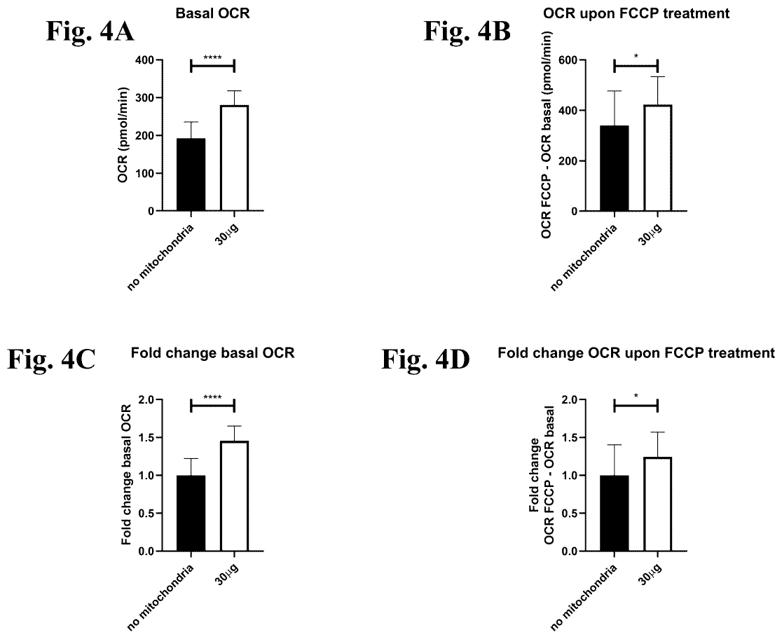

As depicted in FIG. 5A and FIG. 5C, the treatment of CD8⁺ T cells with exogenous mitochondria derived from human cardiac fibroblasts significantly improved mitochondrial activity. Meanwhile, the mitochondrial mass was significantly reduced in CD8⁺ T cells treated with exogenous mitochondria FIG. 5B and FIG. 5D. When combined in a ratio of mean fluorescence intensity of Mitotracker Red CMXRos over Mitotracker Green FM, it revealed an enhanced mitochondrial fitness in CD8⁺ T cells treated with exogenous mitochondria FIG. 5E. Moreover, the improvement in mitochondrial fitness—i.e. the ratio of mitochondrial activity to mitochondrial mass—were shown to be dose-dependent, with significant differences across the treatment groups.

### Example 9: Assessing the Impact of Mitochondria Transplantation on CD8⁺ T Cell Exhaustion

An experiment was performed to determine to what extend the transplantation of exogenous mitochondria into CD8⁺ T cells can mitigate T cell exhaustion.

Procedure
(i) CD8⁺ T cells were isolated from healthy donor and cultivated. The isolation process is described in Example 2.
(ii) CD8⁺ T cells were artificially exhausted in-vitro through repeated CD3/CD28 beads stimulations (ThermoFisher, 111.32D). Three days post T cell activation, CD3/CD28 beads were removed using The Big Easy” EasySep™ Magnet (Stemcell, 18001) and fresh CD3/CD28 beads were added to the CD8⁺ T cells at a ratio of 1 to 1. This step was repeated every two days, three times in a row. T cells were analyzed over time regarding typical signs of exhaustion using anti-human LAG-3 PE/Cyanine7 (Biolegend, 369310) and anti-human TIM-3 APC (Biolegend, 345012) according to the supplier's instructions. A sample of the cells were washed twice with FACS buffer and resuspended in 300p1 before being acquired by a flow cytometer (FACSLyric, BD Biosciences). After 4 stimulations of CD8⁺ T cells with Dynabeads Human T-Activator CD3/CD28 (ThermoFisher, 111.32D), established markers of T cell exhaustion—LAG-3 and TIM-3—were shown to be overexpressed in comparison to CD8⁺ T cells, which received only a single beads-stimulation. To mock collected CD8⁺ T cells from cancer patients, which are rescued from continuous TCR stimulation at the tumor bed, day 10 post activation, the CD3/CD28 beads were removed from the cultured T cells. T cells were transplanted with mitochondria day 11 post activation and 24 h later, CD8⁺ T cells were restimulated with beads to mimic the re-infusion to the patient by ACT. 48 h post restimulation, CD8⁺ T cells were harvested and their exhaustion phenotype was assessed by flow cytometry (e.g., FACSLyric (BD Biosciences)). The expression of exhaustion markers was significantly reduced in exhausted CD8⁺ T cells, which were subsequently treated with exogenous mitochondria. The results confirmed that mitochondria transplantation is effective in mitigating T cell exhaustion in a dose-dependent manner.
(iii) T cell isolation, activation and culture was performed as described in Example 2 for the control cells. The isolation and activation of the cells described in Example 2 was done prior to the cells' exhaustion described in the paragraph (ii) above. 24 hours post mitochondria transplantation, a final re-stimulation of CD8⁺ T cells was performed, prior to measuring the exhaustion markers 72 h post transplantation.
(iv) Mitochondria isolation: Mitochondria were isolated from human cardiac fibroblasts as previously described in Example 1b. The isolated mitochondria were suspended in Homogenizing Buffer of Example 1b and kept on ice until use. Mitochondria quantity, in preparation for varying dosage administration, was measured using a Qubit Fluorometer (ThermoFisher Scientific/Invitrogen), employing the Qubit Protein Assay kit in accordance with the manufacturer's instructions. For the protein concentration measurement, the mitochondria were resuspended in PBS (ThermoFisher, 10010031). The mitochondria dosage is estimated in terms of protein content expressed in g.
(v) Mitochondria Transplantation: CD8⁺ T cells were plated at 0.5 Million cells/mL in a 24 well plate 24 h prior to mitochondria transplantation. When the mitochondria were isolated, CD8⁺ T cells were collected and centrifuged for 5 minutes at 1500 rpm (430×g). The supernatant was discarded and the cells were resuspended in fresh T cell medium at the concentration of 1 Million cell/100 μL. Mitochondria were added to the T cells to obtain a final volume of 200 μL per well of a 24 well plate. The amount of mitochondria with which the T cells were treated was varied, to range between 10 μg to 100 μg. The mitochondria were co-incubated with the CD8⁺ T cells 72 hours before the exhaustion parameters were determined.

Results: Mitigation of Exhaustion by Mitochondria Transplantation

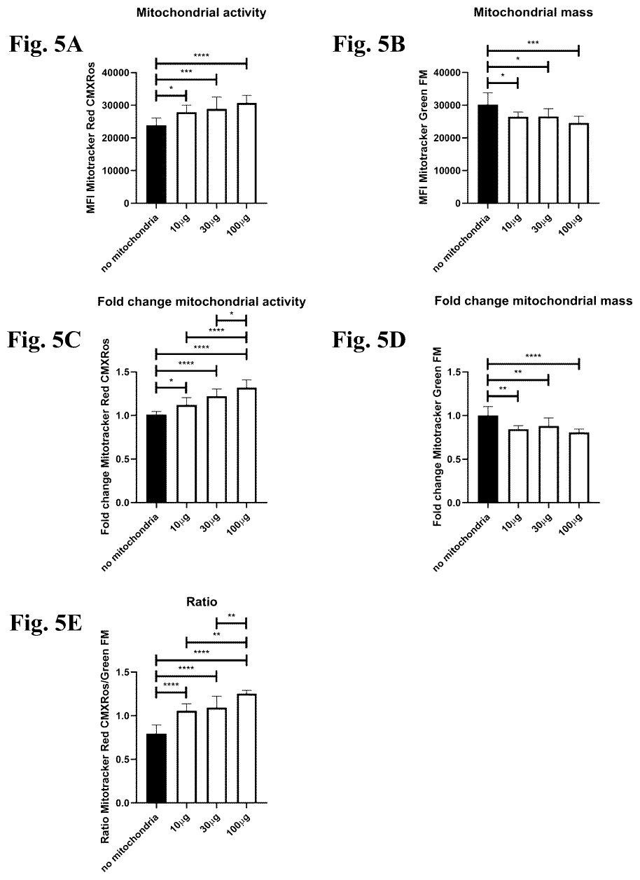

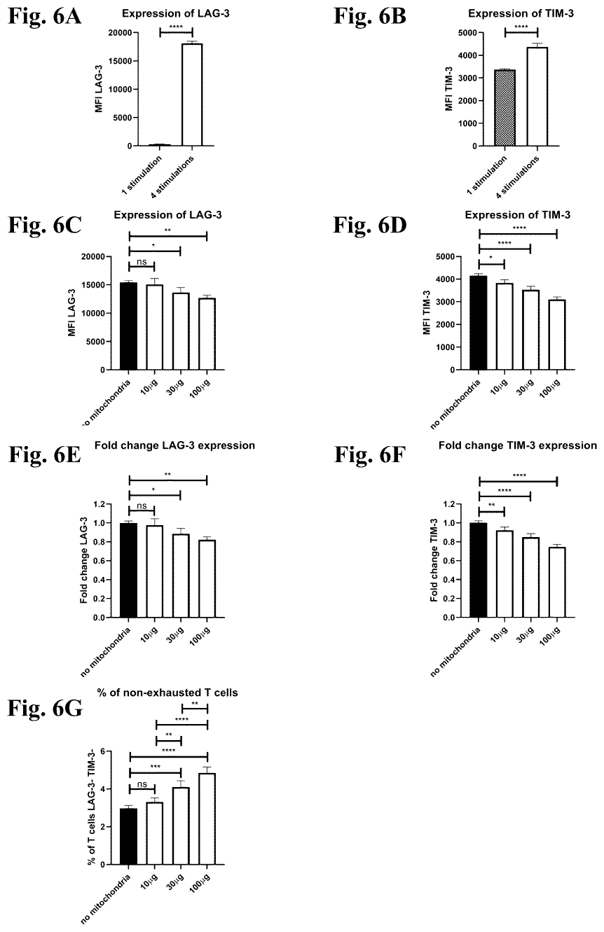

LAG-3 and TIM-3 were overexpressed in CD8⁺ T cells, which were exposed to four rounds of stimulation, when compared to CD8⁺ T cells that received only a single stimulation, as depicted in FIG. 6A and FIG. 6B. As described above, mitochondria were transplanted into the exhausted CD8 T cells in dosages of 10 μg, 30 μg, and 100 μg of protein per 1 Million of CD8⁺ T cells. Subsequently, the cells were re-stimulated with CD3/CD28 beads at a 1 to I ratio. 48 h hours post re-stimulation, the expression of the exhaustion markers was assessed by using anti-human LAG-3 (Biolegend, 369310) and anti-human TIM-3 (Biolegend, 345012) in accordance with the manufacturer's instructions, via flow cytometry, employing a FACSlyric (BD Biosciences). As shown in FIG. 6C-6F, LAG-3 and TIM-3 were significantly reduced, suggesting an improvement in the cells' exhaustion state. This improvement may be predictive of a higher resistance to cancer-induced immunosuppressive mechanisms in vivo. The difference in expression was significant, as indicated. Moreover, the percentage of non-exhausted cells—i.e., CD8⁺ T cells that neither express TIM−⁺3 nor LAG-3—is significantly increased at the dosage levels of 30 μg and 100 μg of mitochondria, as depicted in FIG. 6G. The results confirm that I cell exhaustion is mitigated by transplanting exogenous mitochondria in a dose-dependent manner into T cells.

### Example 10: Impact of In Vitro-Induced Treg Cell Activity Upon Mitochondria Transplantation

Procedure

CD4⁺ T cells are isolated from buffy coats of healthy donors. Peripheral blood mononuclear cells (PBMC) are collected by density gradient centrifugation using Ficoll Paque plus (cytiva, 17144002) according to the manufacturer's instructions. Human CD4⁺ T cells are harvested from the PBMCs using the EasySep™ Human naïve CD4⁺ T Cell Isolation Kit (Stemcell, 19555) and The Big Easy” EasySep™ Magnet (Stemcell, 18001). Isolated naïve CD4⁺ T cells are activated with Dynabeads Human T-Activator CD3/CD28 (ThermoFisher, 111.32D), in a 1 to 1 ratio, in presence of 100 U/mL of recombinant human IL-2 (Peprotech, 200-02). Naïve T cells are culture in serum-free X-Vivo 15 medium (Lonza, BE02-060F) supplemented with 2 mM L-alanyl-L-glutamine (ThermoFisher. 35050061). Treg differentiation is induced by adding to the culture medium 5 ng/mL of TGF-β1 (R&D Systems, 240-B-002) and 10 nM of ATRA (Sigma-Aldrich, R2625) dissolved in DMSO (Schmidt et al., “Comparative Analysis of Protocols to Induce Human CD4+Foxp3+ Regulatory T Cells by Combinations of IL-2, TGF-beta, Retinoic Acid, Rapamycin and Butyrate”, PLOS ONE, 2016). 6 days post culture, induced-Tregs (iTreg) are analyzed for their expression of CD4, CD25, FOXP3 using True-Nuclear Human Treg Flow Kit (Biolegend, 320027) according to the manufacturer's instructions. iTreg cells are transplanted with various doses of mitochondria in a range of 10 μg to 100 μg per million of cells. The suppressive functions of iTregs are evaluated upon mitochondria transplantation on cultured T cells as described in Example 2.

Results

Upon mitochondria transplantation iTregs display an enhanced activity illustrated by more potent suppressive functions on culture T cells.

### Example 11: T Cell Restimulation and Enhancement

The effect of co-incubation (enhancement) with mitochondria on restimulation/enhancement, such as growth enhancement, of T cells is explored. The CD8⁺ T cells remain cytotoxic after restimulation and enhancement, such growth enhancement. Restimulation/enhancement may be necessary when a reduced rate of proliferation is observed.

For restimulation experiments, 5×10⁴ purified T cells are added in 100-200 μL medium to each well in a 96-well tissue culture plate. The cells are incubated in advanced RPMI Medium 1640 with 2 mM L-Glutamine, 10% FCS/FBS and 100 U/mL penicillin/streptomycin. Alternatively, Cancer Stem Premium™ (ProMab Biotechnologies, Richmond, CA) with 100 U/mL penicillin/streptomycin can be used. Mitochondria are added (10⁶-10⁷/well) to the cells and incubated in a humidified CO₂ incubator at 37° C. for 1-24 hours. For T-cell activation, 2 μL pre-washed and resuspended CD3/CD28 Macrobeads™ (ProMab) is added to obtain a bead-to-cell ratio of 1:1. The activated T cells are harvested and used directly for further analyses. Following the co-incubation with mitochondria, the CD8⁺ T cells after restimulation and enhancement have an improved expansion.

### Example 12: Mitochondrial Transfer Increases the Killing Capacity of CAR-T Cells

To assess the impact of mitochondria transplantation on CAR-T cell killing capacity, a FACS-based killing assay is performed 24 h post transplantation. CAR-T cells (CD19-41BB-CD3z, PMC746) are purchased from ProMab Biotechnologies (Richemond, CA 94806). To measure the percentage of target cells expressing an early maker of apoptosis, in transition to apoptosis or apoptotic, respectively: Annexin V+, AnnexinV+/PI+ or PI+ percentages are evaluated. CAR-T cells transplanted or not are plated at an effector to target ratio of 5 to 1. CAR-T cells are co-incubated for 4 h with target cells Daudi (ATCC CCL-213), a B lymphoblast cell line expressing CD19. Post co-incubation, the cells are collected and stained with anti-human CD8 PerCP-Cy5.5 (Stemcell, 60022PS) and anti-human CD3 APC (Stemcell, 60011AZ) for 20 minutes at 4° C. After a wash with FACS buffer, the cells are stained with Annexin V FITC (Biolegend, 640914) and Propidium Iodide (PI, Biolegend, 640914) for 15 minutes at room temperature according to the supplier's instructions and acquired on a FACSLyric (BD Biosciences).

Procedure
(i) CAR-T cell culture is performed as described in Example 2.
(ii) Mitochondria isolation: Mitochondria are isolated from Human Cardiac Fibroblast (HCF) according to the procedure described in Example 1b.
(iii) Quantification of isolated mitochondria: The mitochondria dosage is estimated in terms of protein content expressed in g, according to the procedure described in Example 1b.
(iv) CAR-T cell transplantation according to the procedure of Example 3. An amount of 30 μg or 100 μg of mitochondria is transplanted in the CAR-T cells.

Mitochondria transplantation enhances the killing capacity of CAR-T cells. Percentages of target cells expressing an early marker of apoptosis, in transition to apoptosis or a late marker of apoptosis are increased upon co-incubation with transplanted CAR-T cells.

### Example 13: CAR-T Cell Culture and Labeling

Human anti-CD19scFv-FLAG-CD28-CD3ζ (ProMab) T cells (CAR T cells) are cultured in complete X-Vivo 10 (Lonza, Morristown, NJ) medium supplemented with IL-2 (10 ng/mL, BioLegend, Dedham, MA) at a concentration of 5×10⁵ cells/mL. To identify the CD4⁺ and CD8⁺ subsets, 1×10⁵ CAR-T cells are pelleted and resuspended in 100 μL of PBS containing anti-human CD4 FITC (Miltenyi Biotec) and anti-human CD8 Alexa Fluor 647 (BD Biosciences, Billerica, MA). The labeling reaction is incubated for 15 minutes at room temperature in the dark. The cells are then rinsed twice in PBS and once in complete RMPI medium (Thermo Fisher Scientific) with 10% FBS (v/v; Zenbio, Research Triangle, NC). Raji cells (ATCC, Manassas, VA) are labeled with Vybrant DiD (Thermo Fisher Scientific) following the manufacturer's instructions. Briefly, the Raji cells are resuspended at a density of 1×10⁶ cells/mL in serum-free RPMI containing 5 μL/mL of Vybrant DiD solution and incubated for 20 minutes at 37° C. The labeled cells are then washed three times in complete RMPI medium and kept at 4° C. before use.

To prepare CAR T samples enhanced with exogenous mitochondria, CAR T cells are added in 100-200 μL medium to each well in a 96-well tissue culture plate. The cells are incubated in complete X-Vivo 10 (Lonza) medium supplemented with IL-2 (10 ng/mL, BioLegend). Mitochondria are added (10⁶-10⁷/well) to the cells and incubated in a humidified CO₂ incubator at 37° C. for 1-24 hours.

### Example 14: CAR-T Cell Stimulation with Target Tumor Cells

The effect of co-incubation (enhancement) of CAR T cells with mitochondria on CAR T cell stimulation in a tumor model is explored.

CAR-T cells, either enhanced with exogenous mitochondria as described above or not, and Raji cells (5×10⁴ cells/mL concentration) are incubated for 6 h in a round bottom well plate or a flat bottom plate. Post incubation, the co-cultured cells are collected and centrifuged at 1000 rpm (190×g) to remove the debris and dead cells. Afterward, Raji cells are separated from co-culture by positive selection using a protocol of B cell selection with MagCellect (R&D Systems, Minneapolis, MN). Briefly, CAR-T: Raji cell co-culture is centrifuged and the cell pellet is resuspended in serum-free RPMI medium and labeled with anti-CD19 biotinylated antibodies (BioLegend). Following manufacturer's protocol, MagCellect Streptavidin Ferrofluid is added to the solution and the mixture is incubated for the recommended amount of time according to the product manual. The Streptavidin Ferrofluid beads bound to the anti-CD19 biotinylated Raji cells are collected on the side of the test tube by applying a magnet and then the CAR-T cells are pipetted out of the reaction. This procedure is repeated twice to make sure that most Raji cells are separated. The isolated CAR-T cells are then added to the microwell array device for mRNA capture and transcriptome sequencing. Unstimulated CAR-T cells (5×10⁴ cells/mL) are incubated for 6 h alone before subjected to single-cell RNA sequencing (scRNA-seq).

Relative to non-enhanced or control CAR-T cells, the CAR-T cells enhanced with exogenous mitochondria produce higher levels of both IL-2 and IFN-γ mRNA as well as of TNF-α and Granzyme B, when co-cultured with the target cells. In contrast, co-culture with antigen-negative cells results in little or no cytokine mRNA production.

### Example 15: Mitochondrial Transfer Increases CAR-T Cell Proliferation in Presence of the Target Cells

The effect of co-incubation (enhancement) of CAR T cells with mitochondria on CAR T cell proliferation in response to antigen-specific stimulation is explored.

To measure antigen-specific proliferation, EdU (5-ethynyl-2′-deoxyuridine) is added to CAR-T cells, either enhanced with exogenous mitochondria as described above or not, to a final concentration of 10 μM one hour before the incubation endpoint. After the incubation for 2-4 h, the cells are harvested by centrifugation at 300 g for 5 minutes at 4° C. The cell pellets are incubated at room temperature with Human Fc block for 10 minutes. For cell surface staining, 40 μL of the flow cytometry proliferation panel is added (CD3, CD4, CD8, isotype control; the volume used depends on the conjugate used) and incubated for 30 minutes at 4° C. in the dark. The cells are fixed with 4% paraformaldehyde for 15 minutes at room temperature, followed by an extensive wash with a Triton X-100 based permeabilization buffer. The Click-iT® EdU reaction cocktail is prepared according to the manufacturer's instructions. 100 μL of the permeabilization buffer and 100 μL of Click-iT® EdU reaction cocktail are added to the cell pellet. The mixture is pipetted to a homogenous suspension and then incubated for 30 minutes at 4° C. (acceptable range 2-8° C.) in the dark. The % EdU⁺ cells measured indicates the population of cells that is actively proliferating, and thus synthesizing DNA.

For staining of total cellular DNA, samples are washed once in permeabilization buffer. The DNA staining solution (1 to 20 dilutions with permeabilization buffer) is added to the cells 15 minutes before flow cytometry data acquisition. After an incubation period, 200 μL of permeabilization buffer is added to the cells, and the cells are subsequently analyzed using either a Becton Dickinson (BD) FACSVerse™, BD LSRFORTESSA™, BD FACSCanto™, BD FACSCalibur™, BD™ LSR II or ACEA NovoCyte™ flow cytometer utilizing either FACSuite™, FACSDiva™, or NovoExpress™ software, respectively.

Relative to non-enhanced or control CAR-T cells, the CAR-T cells enhanced with exogenous mitochondria demonstrate higher cell proliferation levels when co-cultured with the target cells.

In contrast, co-culture with antigen-negative cells results in little or no CAR-T cell proliferation.

### Example 16: Cytokine Release Analysis

The effect of co-incubation (enhancement) of CAR T cells with mitochondria on CAR T cell cytokine production in response to antigen-specific stimulation is explored.

CAR-T cells, either enhanced with exogenous mitochondria as described above or not, are prepared and stimulated as described above. The cytometric bead array human Th1/Th2/Th17 kit (BD Biosciences) is used to measure IL-2, IL-4, IL-6, IL-10, TNFα, IFN-7 and IL-17A protein levels in each sample. Cytokine standards are prepared by serial dilution of lyophilized human Th1/Th2/Th17 cytokines according to the manufacturer's instructions. Flow cytometry is used to detect the cytokine levels.

Relative to non-enhanced or control CAR-T cells, the CAR-T cells enhanced with exogenous mitochondria produce higher cytokine levels when co-cultured with the target cells. In contrast, co-culture with antigen-negative cells results in little or no cytokine production.

### Example 17: Mitochondrial Transfer Increases Cytotoxic Activity of CAR-T Cells

The effect of co-incubation (enhancement) of CAR T cells with mitochondria on CAR T cell cytotoxic activity in a tumor model is explored.

The CD19⁺Raji target cells, cultivated under standard conditions, are collected by centrifugation, washed twice, and re-suspended in RPMI-1640 medium. One mL containing 2.5×10⁶ cells is mixed with calcein-AM (Life Technologies) to a final concentration of 10 μM and incubated at 37° C. for 30 minutes. The target cells are washed three times in RPMI-1640/10% fetal bovine serum (FBS) and the cell density is adjusted to 3×10⁵ cells/mL. Human anti-CD19scFv-FLAG-CD28-CD3ζ (Promab) T cells are cultured in complete X-Vivo 10 (Lonza) medium supplemented with IL-2 (10 ng/ml, BioLegend) at a concentration of 5×10⁵ cells/mL. The CAR-T cells are pelleted and resuspended in fresh medium at a density 6×10⁶ cells/mL. Fifty μL of the target and different amounts of effector CAR-T cells (with or without exogenous mitochondria prepared as described above) are mixed in the same wells of a 96-well microtiter plate thus providing a range of effector-to-target (E:T) cell ratios and the plate is incubated at 37° C. for 4 hrs. After 3 hrs and 45 minutes incubation, 20 μL 0.9% Triton X-100 is added to the control wells to achieve complete lysis of the target cells (referred as maximal lysis). One hundred μL supernatant of each sample is then transferred into a black microtiter plate and the fluorescence (excitation at 488 nm, emission at 518 nm) is recorded using a Tecan M200 plate reader. Each experiment is carried out in quadruplicate. The fluorescence intensity of the samples without CAR-T cells is subtracted as a background and the percentage of specific lysis in samples with antibodies is calculated. To determine ED₅₀ values (effective doses of effector cells leading to 50% maximal killing), the dose-response curves are computed by a nonlinear regression analysis and a three-parameter fit model ‘log [agonist] vs. response’ using the software program PRISM (GraphPad, San Diego, CA).

Alternatively, cytotoxicity is assessed using the Pierce LDH Cytotoxicity Assay kit (Thermo Fisher Scientific). The effector cells and target cells are mixed at three ratios, i.e., 1:1, 5:1, and 10:1. Target and effector cells are incubated for 6 h in complete RPMI medium at a final concentration of 7×10⁵ cells/mL. LDH release is measured in the supernatant according to manufacturer's instructions. Maximum LDH release is obtained by incubating target cells in the provided 10× lysis buffer. Target cell cytotoxicity is calculated using the following formula: % of cytotoxicity=100×[(CAR-T: target cells−CAR-T cells alone−target cells alone)/(maximum target cell lysis−target cells alone without lysis buffer)].

Relative to non-enhanced or control CAR-T cells, the CAR-T cells enhanced with exogenous mitochondria demonstrate higher cytotoxicity (lower ED₅₀ values) when co-cultured with the target cells. In contrast, co-culture with antigen-negative cells results in little or no cell killing.

### Example 18: Mitochondrial Transfer Increases Anti-Tumor Activity of CAR-T Cells In Vivo

Human T cells expressing anti-CD19 CAR-T constructs (anti-CD19scFv-FLAG-CD28-CD3ζ, Promab), either enhanced with exogenous mitochondria or not, are evaluated in mouse xenograft models of B-cell lymphoma.

Lymphoma Model

Nine-week-old female NOD/SCID mice (non-obese diabetic; deficient for T cells, macrophages and NK cells; Taconic, Denmark) are subcutaneously (s.c.) injected with human Burkitt's lymphoma CD19⁺ Raji cells (2.5×10⁶ cells/mouse). Animals are randomized into treatment groups when the tumors reached the size of 60-100 mm³; 5-8 mice per group with equal tumor size are selected for the treatment. The animals received i.v. injections of 10⁷ mock-transduced T cells, or anti-CD19 CAR-T cells, or mitochondria-enhanced anti-CD19 CAR-T cells. Tumor size is measured in two dimensions with a caliper-like instrument. Individual tumor volumes (V) are calculated by the formula V=0.56×(length+width)². Upon reaching the humane endpoint with a tumor volume of 1,500 mm³, the animals are sacrificed by cervical dislocation. The Kaplan-Meier survival plots are generated using the software program PRISM (GraphPad) and the survival curves are compared using a log-rank (Mantel-Cox) test.

Leukemia Model

Eight-week-old male NSG (NOD/SCID gamma mouse; deficient for T cells, B cells and NK cells) mice purchased from Jackson Laboratories are housed in the vivarium in sterile cages. Raji/Luc-GFP cells (10⁶) in 100 μL PBS are injected i.v. via the lateral tail vein using an insulin syringe (designated as day 0). Luciferase activity is measured on day 6 via bioluminescence imaging to assess tumor burden. On day 7, 10⁷ mock-transduced T cells, anti-CD19 CAR-T cells, or mitochondria-enhanced anti-CD19 CAR-T cells are prepared in 100 μL PBS, and injected i.v. using an insulin syringe. Tumor progression is monitored by bioluminescence imaging using an IVIS imaging system. At day 60, surviving mice are euthanized, spleen and bone marrow cells harvested and re-suspended in a total volume of 2 mL of flow cytometry (FACS) buffer (PBS, supplemented with 2% FCS). Two hundred microliters of the cell suspension are then labeled with PE-anti-huCD3 and APC-anti-huCD45 antibodies, and analyzed by flow cytometry to determine the percentage of human T cells.

Relative to non-enhanced or control CAR-T cells, the CAR-T cells enhanced with exogenous mitochondria demonstrate higher anti-tumor activity (longer median survival) in the treated mice.

### INCORPORATION BY REFERENCE

The entire disclosures of all patent and non-patent publications cited herein are each incorporated by reference in their entireties for all purposes.

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and sub-combinations of the various elements, features, functions, and/or properties disclosed herein. The following claims particularly point out certain combinations and sub-combinations regarded as novel and nonobvious. Inventions embodied in other combinations and sub-combinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1-84. (canceled)

85. A method of increasing proliferative capacity of a human immune cell comprising:
receiving a sample from a human subject comprising the human immune cell;
isolating the human immune cell from the sample;
optionally genetically modifying the human immune cell;
contacting the human immune cell with exogenous, isolated, viable, respiration-competent mitochondria under conditions that induce uptake of the mitochondria into the human immune cell to generate a modified human immune cell;
wherein the modified human immune cell has increased proliferative capacity as compared to the human immune cell.

86. The method of claim 85, wherein the exogenous isolated, viable, respiration-competent mitochondria are intact mitochondria.

87. The method of claim 85, wherein the modified human immune cell has decreased cellular exhaustion as compared to the human immune cell not comprising the respiration-competent mitochondria, and wherein cellular exhaustion is measured through expression of LAG-3 and TIM-3.

88. The method of claim 85, wherein the human immune cell is a T lymphocyte.

89. The method of claim 88, wherein the T lymphocyte is a Regulatory T cell.

90. The method of claim 89, wherein the Regulatory T cell exhibits enhanced suppressive functionality of T cell activity as compared to a Regulatory T cell not comprising exogenous mitochondria.

91. The method of claim 88, wherein the T lymphocyte is a Chimeric Antigen Receptor (CAR) T cell.

92. The method of claim 91, wherein the CAR T cell exhibits increased production of mRNA for IL-2, IFN-7, TNF-α, or Granzyme B, or a combination thereof, when exposed to a target cell as compared to a CAR T cell not comprising exogenous mitochondria.

93. The method of claim 91, wherein the CAR T cell exhibits increased cytokine production when exposed to a target cell as compared to a CAR T cell not comprising exogenous mitochondria.

94. The method of claim 91, wherein the CAR T cell exhibits increased cytotoxicity when exposed to a target cell as compared to a CAR T cell not comprising exogenous mitochondria.

95. The method of claim 91, wherein a mammalian animal with a cancer has a longer median survival rate when treated with the CAR T cell comprising exogenous mitochondria as compared to a CAR T cell not comprising exogenous mitochondria.

96. The method of claim 85, wherein the human immune cell is obtained from a patient in need of treatment for cancer.

97. The method of claim 85, wherein the human immune cell is obtained from a patient in need of treatment for autoimmune disease.

98. The method of claim 85, wherein the exogeneous mitochondria is obtained from a healthy donor.

99. The method of claim 85, wherein the exogeneous mitochondria is obtained from a cultured cell line.

100. The method of claim 85, wherein the exogeneous mitochondria is obtained from human cardio fibroblasts.

101. A pharmaceutical composition comprising a human immune cell comprising exogenous mitochondria and a pharmaceutically acceptable carrier;
wherein the exogenous mitochondria is present in the human immune cell in an amount effective to enhance immune cell survival and/or activity of the human immune cell;
and wherein the exogenous mitochondria is obtained from human cardiac fibroblasts;
and wherein the proliferative capacity of the human immune cell is increased by the presence of the exogenous mitochondria.

102. The pharmaceutical composition of claim 101, wherein the human immune cell is treated with 30 ug of mitochondria as measured by the mass of mitochondrial protein.

103. The pharmaceutical composition of claim 101, wherein the human immune cell is treated with 100 ug of mitochondria as measured by the mass of mitochondrial protein.

104. A method of making a pharmaceutical composition of claim 101, the method comprising:
a) obtaining a sample from a patient in need of treatment;
b) isolating and/or enriching the human immune cell from the sample;
c) culturing the human immune cells of step (b) with the mitochondria under conditions that induces uptake of the mitochondria by the human immune cell; and
d) formulating the human immune cell of step (c) with a pharmaceutically acceptable carrier;
wherein the human immune cells are optionally genetically modified before, during or after step (c).
